(19) United States
(12) Patent Application Publication
(10) Pub. No.: US 2006/0267004 A1
(43) Pub. Date: Nov. 30, 2006

(54) Compounds comprising a linear series of five fused carbon rings, and preparation thereof

(76) Inventors: Alexander Graham Fallis, Ottawa (CA); Christophe Benard, Verrieres Le Buisson (FR); Kelly Vancrey, Bright's Cove (CA); Zhe Geng, Ottawa (CA)

Correspondence Address: KIRBY EADES GALE BAKER, BOX 3432, STATION D, OTTAWA, ON, K1P 6N9, CA

(21) Appl. No.: 11/439,309

(22) Filed: May 24, 2006

Related U.S. Application Data
(60) Provisional application No. 60/684,948, filed on May 27, 2005.

(51) Int. Cl. H01L 51/00; C07C 13/28

(52) U.S. Cl. 257/40; 552/284; 313/504; 585/422

(57) ABSTRACT

The present application discloses methods for the production of organic compounds comprising a linear series of five fused carbon rings. Such compounds are useful in the production of electronic components, devices and materials. For example the methods disclosed permit the production of 2,9- and 2,10-disubstituted pentacene compounds that present particularly advantageous properties for the manufacture of semiconductor materials, or ink jet fabrication, and may be used in devices such as for example thin film transistors and solar cells. Also disclosed are compounds that are excellent candidates for use in the manufacture of semiconductor materials, and other components of electronic systems, by virtue of their solubility, crystal packing geometries, and electronic properties.

### CROSS-REFERENCE TO RELATED APPLICATION

This application claims the benefit of U.S. Provisional Application No. 60/684,948 filed May 27, 2005 entitled “Compounds comprising a linear series of five fused carbon rings, and preparation thereof”.

### FIELD OF THE INVENTION

The present invention relates to the field of pentacene compounds. More specifically, the present invention relates to compounds comprising a linear series of five fused carbon rings (e.g. 2,9- and 2,10-disubstituted pentacenes), their production and use in semiconductor materials and organic thin film electronic devices.

### BACKGROUND TO THE INVENTION

Semiconductors are materials that have electronic properties between electrical insulators and electrical conductors. The efficiency of a semiconducting material is determined by how easily the electrons and electron ‘holes’ can move through the material—i.e. the electron and hole mobilities (μₑ or μₕ). Highly conjugated organic compounds have overlapping atomic orbitals that form valence and conducting bands similar to metals. Organic semiconductors do not have the same electron or hole mobilities as single-crystalline silicon, but they are advantageous during fabrication as solution processing techniques such as lithography can be used.

Silicon and gallium arsenide semiconductors, silicon dioxide insulators, and metals such as aluminum and copper have dominated the semiconductor industry for many years. More recently, however, organic thin-film transistors (OTFTs) have presented an alternative to the traditional thin-film transistors based on inorganic materials. For example, research efforts have focused on linear acenes (including tetracene and pentacene), thiophene oligomers (including α-sexithiophene), regioregular polythiophenes, copper phthalocyanines and naphthalenebisimides as candidates for organic semiconductors (Katz H. E. et al. Acc Chem Res (2001), 34, 359). Of these, pentacene exhibits the best electron and hole mobilities. Charge-carrier mobility values of 1.5 cm²V⁻¹ s⁻¹, on/off current ratios greater than 10⁸, and sub-threshold voltages of less than 1.6 V have been reported for pentacene-based transistors. Therefore, the charge-carrier mobility values for pentacenes are comparable or even superior to those of amorphous silicon-based devices.

A rapid two-step synthesis for pentacene was reported in 1972, as shown in Scheme 1, and pentacene was found to be both light and air sensitive (Goodings E. P. et al. J Chem Soc, Perkin I (1972), 1310). However, more problematic is the virtual insolubility of pentacene in common organic solvents, thereby preventing solution-based processing (Mayer zu Heingdorf F.-J. et al. Nature (2001) 412, 517). As a result, pentacene must generally be deposited from the vapor phase by vacuum sublimation in order to achieve maximum performance. The vacuum sublimation method, however, requires expensive equipment and lengthy pump-down cycles.

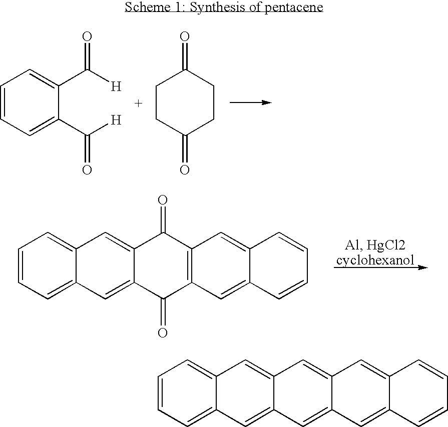

Another disadvantage of pentacene relates to its polymorphic nature, which can have a detrimental influence upon the performance and reproducibility of pentacene-based devices. The alignment or structural order of the pentacene molecules differs for each polymorph or crystallographic phase, and this structural order determines the electronic properties of the device. The crystallographic phase adopted by pentacene depends on the method and conditions under which the crystals are formed. For example, when pentacene is vapor-deposited onto a substrate, a thin film phase is formed. This thin film phase is more effective at transporting charge than pentacene's bulk or single crystal phase, but it is meta-stable. For example, the thin film form of pentacene can be converted to the bulk phase by exposure to solvents such as isopropanol, acetone or ethanol.

More recently, substituted pentacene compounds have been developed that are more soluble in organic solvents, exhibit regular crystal packing, and are better suited for organic processing. For example, international patent publications WO03/028125, and WO03/027050, both published Apr. 3, 2003 and which are incorporated herein by reference, disclose substituted pentacene compounds and methods for their preparation. The substitutions include electron-donating groups and halogen atoms. Such petancene compounds are, at least in preferred embodiments, suited for use in organic semiconductor materials. Particularly useful semiconductor compounds include 2,9- and 2,10-disubstituted pentacenes, which are predicted to exhibit excellent solubility, solid-state packing and Σ-orbital overlap (Anthony, J. E. et al. J Am Chem Soc (2001), 123, 9482; Anthony J. E. et al. Org Lett (2002) 4, 15).

To date, the production of 2,9- and 2,10-disubstituted pentacenes has been difficult to achieve. International patent publication WO03/027050 discloses a method for preparing pentacene derivatives comprising the step of cyclizing at least one substituted bis(benzyl)phthalic acid to form the corresponding substituted pentacenedione by using an acid composition comprising trifluoromethanesulphonic acid, wherein the bis(benzyl)phthalic acid is selected from: 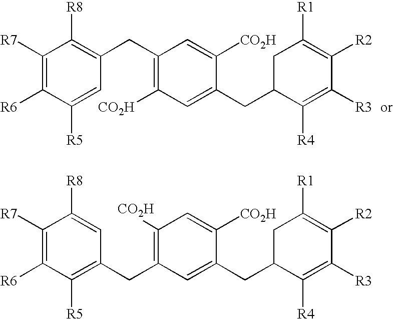
each R representing an electron-donating group, a halogen atom, or a hydrogen atom. In preferred embodiments, the method is suitable for generating a 2,9- or 2,10-disubstituted pentacene 5,7 or 5,12-dione, which can undergo reduction and dehydration to generate the corresponding disubstituted pentacene.

There remains a continuing need to develop novel pathways for the production of compounds comprising a linear series of five fused carbon rings, such as for example 2,9- and 2,10-disubstituted pentacene compounds, and corresponding pentacene derivatives. Moreover, there remains a need to develop methods that are better suited for large-scale production of a broad range of pentacene derivatives, and other compounds comprising a linear series of five fused carbon rings, within minimal cost. New pathways are desired to present opportunities to develop new classes of pentacene derivatives, for example with alternative substitutions either on the A and E rings, or the other rings of the five fused carbon ring core structure.

### SUMMARY OF THE INVENTION

It is one object of the present invention, at least in preferred embodiments, to provide a method for producing compounds comprising a core structure including a linear series of five fused carbon rings.

It is another object of the present invention, at least in preferred embodiments, to provide intermediates suitable for use in the production of pentacene derivatives with one or more substitutions on the A and/or the E rings.

It is another object of the present invention, at least in preferred embodiments, to provide compounds suitable for use in electronic devices, for example in thin film transistors, or for other use as a semiconductor, or for use in inkjet fabrication.

It is another object of the present invention to provide novel compounds comprising a linear series of five fused carbon rings including, but not limited to, novel pentacenes.

Through significant inventive ingenuity, the inventors of the present invention have developed novel methods for the synthesis of organic compounds comprising for example a linear series of five fused carbon rings. Such compounds may include, but are not limited to, benzoquinones and pentacenes. The methods of the present invention permit facile access to a broad range of compounds comprising the aforementioned five-fused carbon ring core. Such compounds include, for example, pentacenes, which may include a broad range of substituents. For example, the inclusion of acetylene groups (or at least substitutions comprising acetylene linkers) on the A and E rings affords access to compounds that are particularly suited to electronic applications. Moreover, such compounds are amenable to further manipulation, for example to custom design pentacenes having optimal electronic properties. The novel compounds of the present invention are suitable for use in the manufacture of numerous types of electronic devices, including for example thin film transistors and solar cells.

In one aspect the present invention provides for a method for the preparation of a compound comprising at least one linear series of five fused carbon rings, the method comprising the steps of:
(a) providing an unsubstituted or substituted benzoquinone;
(b) providing an unsubstituted or substituted acyclic, cyclic, heterocyclic or ortho-quinodimethane diene;
(c) performing a double or stepwise cycloaddition reaction between the benzoquinone and the diene selected to generate a core structure comprising five fused carbon rings sequentially identified as rings A, B, C, D, and E.

Preferably, the method further comprises the steps of:
(d) performing a ring opening reaction to convert a bridged form of each of rings B and D to an unbridged form; and
(e) optionally performing an aromatization reaction or equivalent on the B, and D rings of the core structure;
   wherein steps (d) and (e) can be performed in any order.

Preferably, the method further comprises the step of:
(d) replacing or adding selected substituents.

Preferably, the method further comprises the step of:
(d) subjecting the compound to reducing conditions to generate a corresponding unsubstituted or substituted pentacene.

Preferably, the method generates isomeric products, and the method further comprises the step of:
(d) separating the isomeric products.

Preferably, the method further comprises the step of:
(d) performing a coupling reaction to link two or more core structures.

It should be noted that any of the additional steps (d) and/or (e) described above can be added to the basic methods of the invention. Moreover, any two or more additional steps can be performed in any order.

Preferably in step (a) the benzoquinone has the general formula I:

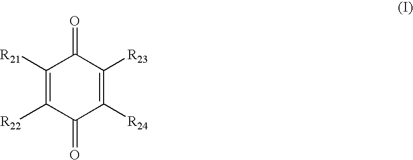

Preferably, in the compounds of formula I, each R group is independently selected from the group consisting of hydrogen, an electron-withdrawing group, and halogen.

Preferably in step (b) the diene compound has the general formula IIa or IIb: 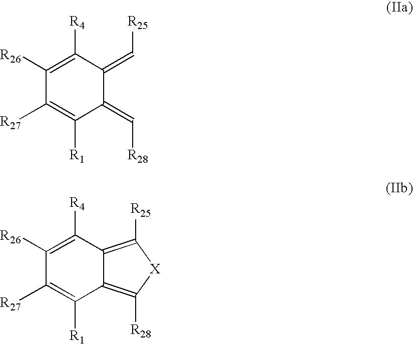
wherein each R group is H or any group that does not interfere with the capacity of the diene to undergo a cycloaddition reaction with benzoquinone, and X is C, O, S, or N.

Preferably, step (c) comprises a double Diels-Alder reaction between the benzoquinone and two diene molecules.

Preferably in step (b) R₂₆ or R₂₇ comprises A-B, wherein A is a protective group, and B is a group to be protected, and wherein the method generates a compound of the formula III: 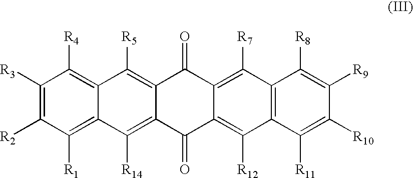
wherein R₂, and R₉ or R₁₀ are A-B, and each remaining R is each independently unsubstituted or substituted. More preferably, the method further comprises replacing each A-B at R₂, and R₉ or R₁₀ with an alternative substituent.

Preferably, when the methods of the invention involve reduction, the step of reduction generates a pentacene compound of formula IV: 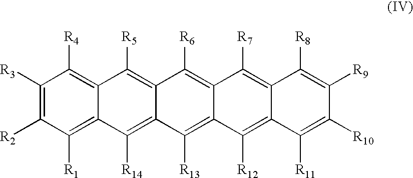
wherein R₂, and R₉ or R₁₀ are A-B, and optionally R₆ and R₁₃ are also A-B, where each A is a protective group and each B is a group to be protected, and each remaining R is each independently unsubstituted or substituted. More preferably, such methods further comprise replacing each A-B is replaced with an alternative substituent. More preferably, R₂, and R₉ or R₁₀ and optionally R₆ and R₁₃ comprise an unsubstituted or substituted group selected from acetylene, alkyl, aryl, heteroaryl, alkenyl, and alkynyl. Most preferably, R₂ and R₉ or R₁₀ comprise acetylene or a linker comprising one or more triple bonds, optionally substituted by halogen and/or triflate.

Preferably, when the methods of the invention comprise coupling, the methods generate a oligomeric compound comprising multiple units of said core structure linked by acetylene groups at the 2, and 9 or 10 positions or the core structures directly linked to each other.

In accordance with the methods of the invention, preferably each A-B comprises Si(R₃₀, R₃₁, R₃₂) wherein each of R₃₀, R₃₁, R₃₂ are independently selected from any group that in conjunction with Si acts to provide a protective group. More preferably, each A-B comprises TMS, TES, TBS, TIPS, diphenyl tertiary butyl, OSi, OH, OTf, OTs, OMs, ONs, NSi, acetylene, phthalocyanine as a metal complex or free ligand, fullerene, Buckminsterfullerene C₆₀R₁₀₀, wherein R₁₀₀ is hydrogen or any substituant, or fullerene linked to the pentacene core via acetylene, or Buckminsterfullerene C₆₀R₁₀₀ linked to the pentacene core via acetylene or phthalocyanine as a metal complex or free ligand linked to the penacene core via acetylene. Indeed, without wishing to be bound by theory it is considered that phthalocyanine pentacenes generated in accordance with the present invention may be particularly suited for use in solar cell or solar panels, or components thereof.

Most preferably, each B is O, S, Se, or N.

Preferably, when the methods of the invention comprise the step of replacing or adding selected substituents, each A-B is replaced with Tf-O, halogen, or a substituent comprising a metal atom selected from Al, B, Cu, Co, Cr, Fe, Li, Mg, Ni, Pd, Pt, Si, Sn, Ti, and Zn. More preferably, the method further comprises replacing each Tf-O with an acetylene group, or a group comprising a linker comprising one or more triple bonds.

Preferably, when the methods of the invention comprise the separation of isomeric products, the step of separating comprises high performance liquid chromatography or fractional crystallization.

Preferably, in the diene compounds of formula IIa or IIb, R₂₅ is a leaving group comprising OAlk, NAlk₂, or halide wherein each Alk comprises an alkyl group of from 1 to 12 carbon atoms.

In another aspect, the present invention provides for a method for the preparation of a pentacene comprising substitutions at least at the 2 positions, and the 9 or 10 position, the method comprising the steps of:
(a) performing a stepwise or double Diels-Alder reaction by reacting a compound of formula IIa or IIb: 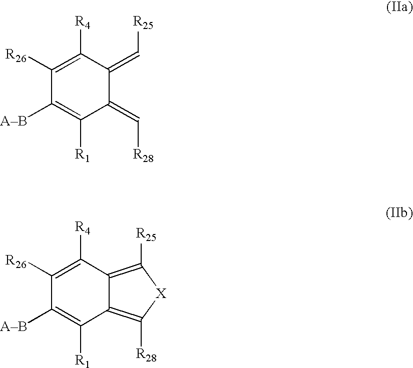
   wherein A is a protective group, B is a group to be protected, and each R group is independent selected from H or a substituent, with a compound of formula I: 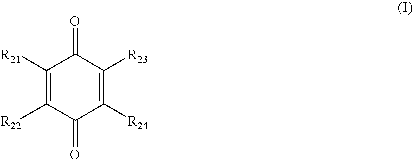
   wherein each R group is independently selected from H or a substituent, and if necessary
(b) optionally performing a ring opening reaction to covert a bridged form of each of rings B and D, to an unbridged form; and
(c) optionally performing an aromatization reaction or equivalent on the B, and D rings of the core structure;
   wherein the method generates a mixture of compounds of formula V and VI: 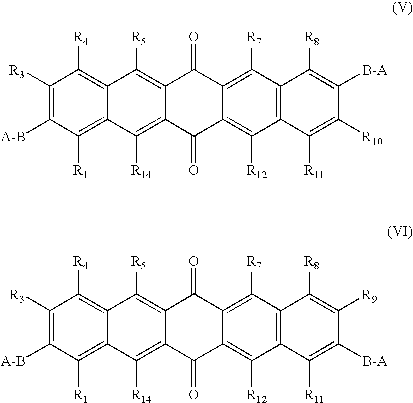
wherein A is a protective group, B is a group to be protected, and each R group is independent selected from H or a substituent.

Preferably, the method further comprises the step of:
(c) separating the compounds of formula (V) and formula (VI), and selecting the compound of formula (V) and/or the compound of formula (VI) for further processing.

Preferably, the method further comprises the step of:
(c) replacing each A or each A-B with an alternative substituent, with or without a linker comprising one or more triple bonds to form a 2,9- and/or a 2,10-disubstituted quinone

Preferably, the method further comprises the step of:
(c) subjecting the 2,9- and/or the 2,10-disubstituted quinone to reducing conditions to generate a pentacene substituted at least in the 2 position, and the 9 or 10 position.

In another aspect, the present invention provides for a compound of the formula III: 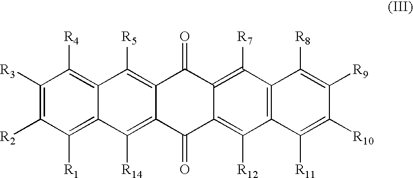
wherein R₁ to R₁₄ are each independently unsubstituted or substituted.

Preferably, the compound of formula III comprises at least one substituent on each of the A and E rings of the core structure. More preferably, the compound comprises at least one substituent on each of the A and E rings, and at least one substituent on at least one of the B, C, or D rings of the core structure. More preferably, the compound comprises substituents at least at the 2, and the 9 or 10 positions. Most preferably, the substituents at the 2, and the 9 or 10 positions are acetylene groups, or are each attached to the core structure via a linker comprising one or more triple bonds. Preferably, in accordance with the compound of formula III each substituent is independently selected from hydroxyl, alkyl, alkoxy, alkenyl, alkynyl, aryl, heteroaryl, acetylene, halogen, and triflate. More preferably, each substituent is substituted by alkyl or halogen.

In another aspect, the present invention provides for a compound of formula IV: 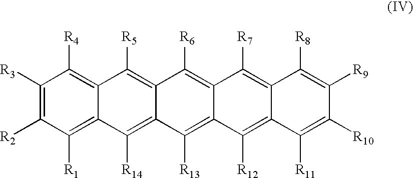
wherein R₂ and R₉ or R₁₀ are A-B, and optionally R₆ and R₁₃ are also A-B where A is a protective group and B is a group to be protected, and each remaining R is independently unsubstituted or substituted.

Preferably, the compounds of formula IV include the proviso that the compounds of formula IV exclude pentacenes comprising alkyl groups at R₂ and R₉ and/or R₁₀.

Preferably, the compounds of formula IV include the proviso that when at least one of R₁, R₂, R₃, R₄, R₈, R₉, R₁₀, and R₁₁ are substituted with an electron-donating substituent, or a halogen, then the compound must include at least one further substituent at R₅, R₆, R₇, R₁₂, R₁₃, or R₁₄.

Preferably, the compound of formula IV comprises at least one substituent on each of the A and E rings, and optionally the C ring, of the core structure. More preferably, the compound comprises at least one substituent on each of the A and E rings, and at least one substituent on at least one of the B, C, or D rings of the core structure. More preferably, the compound comprises substituents at least at the 2, and the 9 or 10 positions and optionally the 6 and 13 positions. Most preferably, the substituents at the 2, and the 9 or 10 positions and optionally the 6 and 13 positions comprise acetylene groups, or are each attached to the core structure via a linker comprising one or more triple bonds. Preferably, in accordance with the compound of formula IV each A-B is replaced by a group independently selected from hydroxyl, alkyl, alkoxy, alkenyl, alkynyl, aryl, heteroaryl, acetylene, halogen, and triflate. More preferably, each A-B is replaced by a group comprising alkyl or halogen.

In another aspect, the present invention provides for the use of a compound according to formula III in the manufacture of a material suitable for use in ink-jet fabrication or as a component of an electronic device. Preferably the use is for the manufacture of a component selected from the group consisting of an Organic Thin Film Semiconductor (OTFS), an Organic Field-Effect Transistor (OFET), an Organic Light Emitting Diode (OLED), a radio-frequency identification tag (RFID), a biosensor, a solar cell, and a component for solar energy conversion.

In another aspect, the present invention provides for the use of a compound according to formula IV in the manufacture of a material suitable for use in ink-jet fabrication or as a component of an electronic device. Preferably the use is in the manufacture of a component selected from the group consisting of an Organic Thin Film Semiconductor (OTFS), an Organic Field-Effect Transistor (OFET), an Organic Light Emitting Diode (OLED), a radio-frequency identification tag (RFID), a biosensor, a solar cell, and a device for solar energy conversion.

Preferably the compound of formula IV is suitable for use as a semiconductor.

In another aspect the present invention provides for semiconductor material derived from processing of the compound of formula III.

In another aspect the present invention provides for an electronic device comprising a component selected from an Organic Thin Film Semiconductor (OTFS), an Organic Field-Effect Transistor (OFET), an Organic Light Emitting Diode (OLED), a radio-frequency identification tag (RFID), a biosensor, a solar cell, and a component for solar energy conversion, wherein said component comprises the semiconductor material derived from processing the compound of formula III.

In another aspect, the present invention provides for an electronic device comprising the semiconductor material derived from processing the compound of formula III.

In another aspect the present invention provides for semiconductor material derived from processing of the compound of formula IV.

In another aspect the present invention provides for an electronic device comprising a component selected from an Organic Thin Film Semiconductor (OTFS), an Organic Field-Effect Transistor (OFET), an Organic Light Emitting Diode (OLED), a radio-frequency identification tag (RFID), a biosensor, a solar cell, and a component for solar energy conversion, wherein said component comprises the semiconductor material derived from processing the compound of formula IV.

In another aspect, the present invention provides for an electronic device comprising the semiconductor material derived from processing the compound of formula IV.
In another aspect, the present invention provides for a method of generating a Diels-Alder reaction adduct of formula VII: 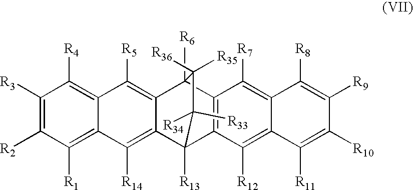
   wherein each of R₁ to R₁₄ are as previously described, and each of R₃₃ to R₃₆ are preferably electron withdrawing groups etc.: by reaction of the compound of formula IV as previously defined, with a dienophile (such as for example sulfur dioxide, alkene dienophile, acyclic dienophile, cyclic dienophile, heterocyclic dienophile, or heteroatom dienophile).

In another aspect, the present invention provides for a method of generating a compound of formula IV as previously defined, the method comprising the step of:
causing the adduct of formula VII as previously defined to undergo thermolysis to regenerate the compound of formula IV.

In another aspect, the present invention provides for a compound of formula VII as previously defined.

In another aspect, the present invention provides for a method of generating a compound of formula VIIIa or VIIIb: 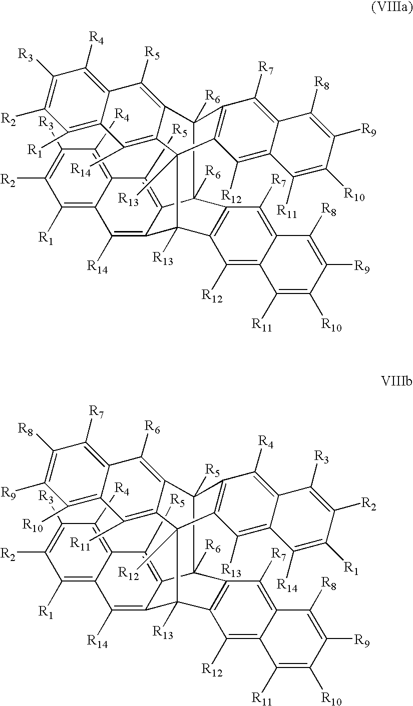
the method comprising the step of: photochemical dimerization of the compound of formula IV as previously defined.

In another aspect, the present invention provides for a method of generating a compound of formula IV as previously defined, by causing the compound of VIIIa and/or VIIIb as previously defined to undergo thermolysis.

In another aspect, the present invention provides for a compound of formula VIIIa or VIIIb as previously defined.

Definitions:

Numbering scheme for pentacenes: Compounds with fused aromatic ring systems are commonly given a numbering sequence in which each carbon atom that is amenable to substitution is numbered. (See, for example, James E. Banks, NAMING ORGANIC COMPOUNDS: A PROGRAMMED INTRODUCTION TO ORGANIC CHEMISTRY, Saunders College Publishing, p. 124, PA (1976).) The numbering sequence that is generally used for pentacene, for example, is shown below.

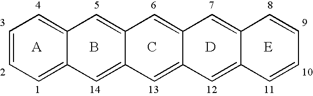

The location of a substituent on such a compound is commonly specified by reference to the number of the carbon atom to, which the substituent is bonded. There is one hydrogen atom bonded to each numbered carbon atom if no substituent is indicated. In general, the rings are identified by a letter A, B, C, and so on as shown above.

Linear Series of Five Fused Carbon Rings:

This expression refers to all compounds comprising a core structure having five fused carbon rings arranged in a linear series. Such compounds include, but are not limited to monoquinones, and pentacenes. Each ring of such compounds may independently be saturated, unsaturated, or aromatic, and be unsubstituted or substituted.

For convenience, the numbering scheme for substituents of all compounds comprising a linear series of five fused carbon rings is generally based upon the pentacene core structure (as discussed above) throughout this specification. However, renumbering of corresponding R groups on products (compared to corresponding substrates) does not necessarily infer that the substituent has been replaced.

Reduction/Reducing Conditions:

The term “reduction” or “reducing conditions” refers to any form of reaction that results in (i) the acceptance of one or more electrons by an atom or ion, (ii) the removal of oxygen from a compound, or the addition of hydrogen to a compound. In the context of this application, the terms further encompass reactions involving alcohols such as, for example, Grignard reactions, including for example reduction/addition to carbonyl to generate an alcohol. The terms include addition to generate an alcohol intermediate, which may be followed by aromatization.

Protective Group:

This expression encompasses any form of protective group, including for example those described in Green, T. W. and Wuts P. G. M., “Protective Groups in Organic Synthesis” (3^{rd} ed. 1999) published by John Wiley ad Sons Inc. Preferably, the protective groups of the present invention are encompassed by A-B, wherein A is a protective group and B is a group to be protected. A-B can include, but is not limited to, OSi, OH, OTf, OTs, OMs, ONs, NSi, and acetylene groups, or groups comprising a linker have at least one triple carbon-carbon bond. A-B therefore includes OH (wherein H can be considered a form of “protecting group”). In preferred embodiments, when B (the group to be protected) includes O or N then A can be silyl, hydrogen or sulfonate alkyl, perfluoroalkyl, or aryl. In other preferred embodiments, where B includes a carbon or hetero atom, then A can be silyl, hydrogen or sulfonate alkyl, perfluoroalkyl or aryl.

Acetylene:

Acetylene groups encompass, at least in preferred embodiments, any group comprising at least one triple carbon bond, or a group comprising a linker comprising at least one triple carbon bond.

Preferably: unless stated otherwise the use of the terms “preferably” and “preferred” refer to preferred features of only the broadest embodiments of the invention.

Additional Chemical Terms

The term “carbo”, “carbyl,” “hydrocarbon” and “hydrocarbyl,” as used herein, pertain to compounds and/or groups which have only carbon and hydrogen atoms.

The term “hetero,” as used herein, pertains to compounds and/or groups which have at least one heteroatom, for example, multivalent heteroatoms (which are also suitable as ring halide) such as boron, silicon, nitrogen, phosphorus, oxygen, and sulfur, and monovalent heteroatoms, such as fluorine, chlorine, bromine, and iodine.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “ring,” as used herein, pertains to a closed ring of from 3 to 10 covalently linked atoms, more preferably 3 to 8 covalently linked atoms.

The term “aromatic ring,” as used herein, pertains to a closed ring of from 3 to 10 covalently linked atoms, more preferably 5 to 8 covalently linked atoms, which ring is aromatic.

The term “heterocyclic ring,” as used herein, pertains to a closed ring of from 3 to 10 covalently linked atoms, more preferably 3 to 8 covalently linked atoms, wherein at least one of the ring atoms is a multivalent ring heteroatom, for example, nitrogen, phosphorus, silicon, oxygen, and sulfur, though more commonly nitrogen, oxygen, and sulfur.

The term “alicyclic,” as used herein, pertains to compounds and/or groups which have one ring, or two or more rings (e.g., spiro, fused, bridged), wherein said ring(s) are not aromatic.

The term “aromatic,” as used herein, pertains to compounds and/or groups which have one ring, or two or more rings (e.g., fused), wherein at least one of said ring(s) is aromatic.

The term “heterocyclic,” as used herein, pertains to cyclic compounds and/or 10 groups which have one heterocyclic ring, or two or more heterocyclic rings (e.g., spiro, fused, bridged), wherein said ring(s) may be alicyclic or aromatic.

The term “heteroaromatic,” as used herein, pertains to cyclic compounds and/or groups which have one heterocyclic ring, or two or more heterocyclic rings (e.g., 15 fused), wherein said ring(s) is aromatic.

Substituents

The phrase “optionally substituted,” as used herein, pertains to a parent group which may be unsubstituted or which may be substituted.

Unless otherwise specified, the term “substituted,” as used herein, pertains to a parent group which bears one or more substituents. The term “substituent” is used herein in the conventional sense and refers to a chemical moiety which is covalently attached to, appended to, or if appropriate, fused to, a parent group. A wide variety of substituents are well known, and methods for their formation and introduction into a variety of parent groups are also well known.

In one preferred embodiment, the substituent(s) are independently selected from: halo; hydroxy; ether (e.g., C₁₋₇alkoxy); formyl; acyl (e.g., C₁₋₇alkylacyl, C₅₋₂₀arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇alkylthio); sulfonic acid; sulfonate; sulfone; sulfonyloxy; sulfinyloxy; sulfamino; sulfonamino; sulfinamino; sulfamyl; sulfonamido; C₁₋₇alkyl (including, e.g., C₁₋₇haloalkyl, C₁₋₇hydroxyalkyl, C₁₋₇carboxyalkyl, C₁₋₇aminoalkyl, C₅₋₂₀aryl-C₁₋₇alkyl); C₃₋₂₀heterocyclyl; or C₅₋₂₀aryl (including, e.g., C₅₋₂₀carboaryl, C₅₋₂₀heteroaryl, C₁₋₇alkyl-C₅₋₂₀aryl and C₅₋₂₀haloaryl)).

In one preferred embodiment, the substituent(s) are independently selected from:

—F, —Cl, —Br, and —I;

—OH;

—OMe, —OEt, —O(tBu), and —OCH₂Ph;

—SH;

—SMe, —SEt, —S(tBu), and —SCH₂Ph;

—C(═O)H;

—C(═O)Me, —C(═O)Et, —C(═O)(tBu), and —C(═O)Ph;

—C(═O)OH;

—C(═O)OMe, —C(═O)OEt, and —C(═O)O(tBu);

—C(═O)NH₂, —C(═O)NHMe, —C(═O)NMe₂, and —C(═O)NHEt;

—NHC(═O)Me, —NHC(═O)Et, —NHC(═O)Ph, succinimidyl, and maleimidyl;

—NH₂, —NHMe, —NHEt, —NH(iPr), —NH(nPr), —NMe₂, —NEt₂, —N(iPr)₂, —N(nPr)₂, —N(nBU)₂, and —N(tBu)₂;

—CN;

—NO₂;

-Me, -Et, -nPr, -iPr, -nBu, -tBu; —CF₃, —CHF₂, —CH₂F, —CCl₃, —CBr₃, —CH₂CH₂F, —CH₂CHF₂, and —CH₂CF₃;

—OCF₃, —OCHF₂, —OCH₂F, —OCCl₃, —OCBr₃, —OCH₂CH₂F, —OCH₂CHF₂, and —OCH₂CF₃;

—CH₂OH, —CH₂CH₂OH, and —CH(OH)CH₂OH;

—CH₂NH₂, CH₂CH₂NH₂, and —CH₂CH₂NMe₂; and,

optionally substituted phenyl.

The substituents are described in more detail below.

C₁₋₇alkyl: The term “C₁₋₇alkyl,” as used herein, pertains to a monovalent moiety obtained by removing a hydrogen atom from a C₁₋₇hydrocarbon compound having from 1 to 7 carbon atoms, which may be aliphatic or alicyclic, or a combination thereof, and which may be saturated, partially unsaturated, or fully unsaturated.

Examples of (unsubstituted) saturated linear C₁₋₇alkyl groups include, but are not limited to, methyl, ethyl, n-propyl, n-butyl, and n-pentyl (amyl).

Examples of (unsubstituted) saturated branched C₁₋₇alkyl groups include, but are not limited to, iso-propyl, iso-butyl, sec-butyl, tert-butyl, and neo-pentyl.

Examples of saturated alicyclic (also carbocyclic) C₁₋₇alkyl groups (also referred to as “C₃₋₇cycloalkyl” groups) include, but are not limited to, unsubstituted groups such as cyclopropyl, cyclobutyl, cyclopentyl, cyclohexyl, and norbornane, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of (unsubstituted) unsaturated C₁₋₇alkyl groups which have one or more carbon-carbon double bonds (also referred to as “C₂₋₇alkenyl” groups) include, but are not limited to, ethenyl (vinyl, —CH═CH₂), 2-propenyl (allyl, —CH—CH═CH₂) isopropenyl (—C(CH₃)═CH₂), butenyl, pentenyl, and hexenyl.

Examples of (unsubstituted) unsaturated C₁₋₇alkyl groups which have one or more carbon-carbon triple bonds (also referred to as “C₂₋₇alkynyl” groups) include, but are not limited to, ethynyl, and 2-propynyl (propargyl).

Examples of unsaturated alicyclic (also carbocyclic) C₁₋₇alkyl groups which have one or more carbon-carbon double bonds (also referred to as “C₃₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

Additional examples of substituted C₃₋₇cycloalkyl groups include, but are not limited to, those with one or more other rings fused thereto, for example, those derived from: indene (C₉), indan (2,3-dihydro-1H-indene) (C₉), tetraline (1,2,3,4-tetrahydronaphthalene (C₁₀), adamantane (C₁₀), decalin (decahydronaphthalene) (C₁₂), fluorene (C₁₃), phenalene (C₁₃). For example, 2H-inden-2-yl is a C₅cycloalkyl group with a substituent (phenyl) fused thereto.

C₃₋₂₀heterocyclyl: The term “C₃₋₂₀heterocyclyl,” as used herein, pertains to a monovalent moiety obtained by removing a hydrogen atom from a ring atom of a C₃₋₂₀heterocyclic compound, said compound having one ring, or two or more rings (e.g., spiro, fused, bridged), and having from 3 to 20 ring atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms.

For example, the term “C₅₋₆heterocyclyl,” as used herein, pertains to a heterocyclyl group having 5 or 6 ring atoms. Examples of groups of heterocyclyl groups include C₃₋₂₀heterocyclyl, C₃₋₇heterocyclyl, C₅₋₇heterocyclyl.

Examples of (non-aromatic) monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅) piperidine (C₆) dihydropyridine (C₆), tetrahydropyridine (C₆), azepine (C₇);

—O₁: oxirane (C₃) oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₆), and oxathiane (thioxane) (C₆); and,

N₁O₁S₁: oxathiazine (C₆).

Examples of substituted (non-aromatic) monocyclic heterocyclyl groups include saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups.

C₅₋₂₀ aryl: The term “C₅₋₂₀ aryl,” as used herein, pertains to a monovalent moiety obtained by removing a hydrogen atom from an aromatic ring atom of a C₅₋₂₀aromatic compound, said compound having one ring, or two or more rings (e.g., fused); and having from 5 to 20 ring atoms, and wherein at least one of said ring(s) is an aromatic ring. Preferably, each ring has from 5 to 7 ring atoms.

In this context, the prefixes (e.g., C₃₋₂₀, C₅₋₇, C₅₋₆, etc.) denote the number of ring atoms, or range of number of ring atoms, whether carbon atoms or heteroatoms. This particularly applied to substituents of the pentacene core of the compounds generated in accordance with the present invention.

For example, the term “C₅₋₆aryl,” as used herein, pertains to an aryl group having 5 or 6 ring atoms. Examples of groups of aryl groups include C₃₋₂₀aryl, C₅₋₇aryl, C₅₋₆aryl.

The ring atoms may be all carbon atoms, as in “carboaryl groups” (e.g., C₅₋₂₀carboaryl).

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), pyrene (C₁₆), and fullerenes particularly for example C₆₀ (“Bucky Ball”) such as C₆₀H or C₆₀R₁₀₀, wherein R₁₀₀ represents any substituent, particularly those discussed herein. Indeed, 2,9 and 2,10 disubstituted pentacenes substituted with fullerene groups generate dumbbell-shaped molecules that may have particular use in specific embodiments.

Examples of aryl groups which comprise fused rings, at least one of which is an aromatic ring, include, but are not limited to, groups derived from indene (C₉), isoindene (C₉), and fluorene (C₁₃).

Alternatively, the ring atoms may include one or more heteroatoms, including but not limited to oxygen, nitrogen, and sulfur, as in “heteroaryl groups.” In this case, the group may conveniently be referred to as a “C₅₋₂₀heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heterocyclic groups (some of which are also heteroaryl groups) which comprise fused rings, include, but are not limited to:

C₉heterocyclic groups (with 2 fused rings) derived from benzofuran (O₁), isobenzofuran (O₁), indole (N₁), isoindole (N₁), purine (N₄) (e.g., adenine, guanine), benzimidazole (N₂), benzoxazole (N₁O1), benzisoxazole (N₁O₁), benzodioxole (O₂), benzofurazan (N₂O₁), benzotriazole (N₃), benzothiofuran (S1), benzothiazole (N₁S₁), benzothiadiazole (N₂S);

C₁₀heterocyclic groups (with 2 fused rings) derived from benzodioxan (O₂), quinoline (N₁), isoquinoline (N₁), benzoxazine (N₁O₁), benzodiazine (N₂), pyridopyridine (N₂) quinoxaline (N₂) quinazoline (N₂);

C₁₃heterocyclic groups (with 3 fused rings) derived from carbazole (N₁), dibenzofuran (O₁), dibenzothiophene (S₁); and,

C₁₄heterocyclic groups (with 3 fused rings) derived from acridine (N₁), xanthene (O₁), phenoxathiin (O₁S₁), phenazine (N₂) phenoxazine (N₁0₁), phenothiazine (N₁S₁), thianthrene (S₂), phenanthridine (N₁), phenanthroline (N₂) phenazine (N₂).

Heterocyclic groups (including heteroaryl groups) which have a nitrogen ring atom in the form of an —NH— group may be N-substituted, that is, as —NR—. For example, pyrrole may be N-methyl substituted, to give N-methypyrrole. Examples of N-substitutents include, but are not limited to C₁₋₇alkyl, C₃₋₂₀heterocyclyl, C₅₋₂₀aryl, and acyl groups.

Heterocyclic groups (including heteroaryl groups) which have a nitrogen ring atom in the form of an —N═ group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms. Monocyclic examples of such groups include, but are not limited to, those derived from:

C₅: cyclopentanone, cyclopentenone, cyclopentadienone;

C₆: cyclohexanone, cyclohexenone, cyclohexadienone;

O₁: furanone (C₅), pyrone (C₆);

N₁: pyrrolidone (pyrrolidinone) (C₅), piperidinone (piperidone) (C₆), piperidinedione (C₆);

N₂: imidazolidone (imidazolidinone) (C₅), pyrazolone (pyrazolinone) (C₅), piperazinone (C₆), piperazinedione (C₆), pyridazinone (C₆), pyrimidinone (C₆) (e.g., cytosine), pyrimidinedione (CO (e.g., thymine, uracil), barbituric acid (C₆);

N₁S₁: thiazolone (C₅), isothiazolone (C₅);

N₁O₁: oxazolinone (C₅).

Polycyclic examples of such groups include, but are not limited to, those derived from:

C₉: indenedione;

N₁: oxindole (C₉);

O₁: benzopyrone (e.g., coumarin, isocoumarin, chromone) (C₁₀);

N₁O₁: benzoxazolinone (C₉), benzoxazolinone (C₁₀);

N₂: quinazolinedione (C₁₀);

N₄: purinone (C₉) (e.g., guanine).

Still more examples of cyclic groups which bear one or more oxo (═O) groups on ring carbon atoms include, but are not limited to, those derived from:

cyclic anhydrides (—C(═O)—O—C(═O)— in a ring), including but not limited to maleic anhydride (C₅), succinic anhydride (C₅), and glutaric anhydride (C₆);

cyclic carbonates (—O—C(═O)—O— in a ring), such as ethylene carbonate (C₅) and 1,2-propylene carbonate (C₅);

imides (—C(═O)—NR—C(═O)— in a ring), including but not limited to, succinimide (C₅), maleimide (C₅), phthalimide, and glutarimide (C₆);

lactones (cyclic esters, —O—C(═O)— in a ring), including, but not limited to, β-propiolactone, γ-butyrolactone, δ-valerolactone (2-piperidone), and ε-caprolactone;

lactams (cyclic amides, —NR—C(═O)— in a ring), including, but not limited to, β-propiolactam (C₄), γ-butyrolactam (2-pyrrolidone) (C₅), δ-valerolactam (C₆) and ε-caprolactam (C₇);

cyclic carbamates (—O—C(═O)—NR— in a ring), such as 2-oxazolidone (C₅);

cyclic ureas (—NR—C(═O)—NR— in a ring), such as 2-imidazolidone (C₅) and pyrimidine-2,4-dione (e.g., thymine, uracil) (C₆).

The above C₁₋₇alkyl, C₃₋₂₀heterocyclyl, and C₅₋₂₀aryl groups, whether alone or part of another substituent, may themselves optionally be substituted with one or more groups selected from themselves and the additional substituents listed below.

Hydrogen: —H. Note that if the substituent at a particular position is hydrogen, it may be convenient to refer to the compound as being “unsubstituted” at that position.

Halo: —F, —Cl, —Br, and —I.

Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₃₋₂₀hetercyclyloxy group), or a C₅₋₂₀aryl group (also referred to as a C₅₋₂₀aryloxy group), preferably a C₁₋₇alkyl group.

C₁₋₇alkoxy: —OR, wherein R is a C₁₋₇alkyl group. Examples of C₁₋₇alkoxy groups include, but are not limited to, —OCH₃ (methoxy), —OCH₂CH₃ (ethoxy) and —OC(CH₃)₃ (tert-butoxy).

Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam, δ-valerolactam, and ε-caprolactam.

Imino (imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of imino groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Acylhalide (haloformyl, halocarbonyl): —C(═O)X, wherein X is —F, —Cl, —Br, or —I, preferably —Cl, —Br, or Carboxy (carboxylic acid): —COOH.

Ester (carboxylate, carboxylic acid ester, oxycarbonyl): —C(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and C(═O)OPh.

Acyloxy (reverse ester): —OC(═O)R, wherein R is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)NH(CH₃)₂, —C(═O)NHCH₂CH₃, and —C(═O)N(CH₂CH₃)₂, as well as amido groups in which R¹ and R² together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, a C₁₋₇alkyl group, a C₃₋₂₀heterocyclyl group, or a C₅₋₂₀aryl group, preferably a C₁₋₇alkyl group, and R² is an acyl substituent, for example, a C₁₋₇alkyl group, a C₃₋₂₀heterocyclyl group, or a C₅₋₂₀aryl group, preferably a C₁₋₇alkyl group. Examples of acylamido groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

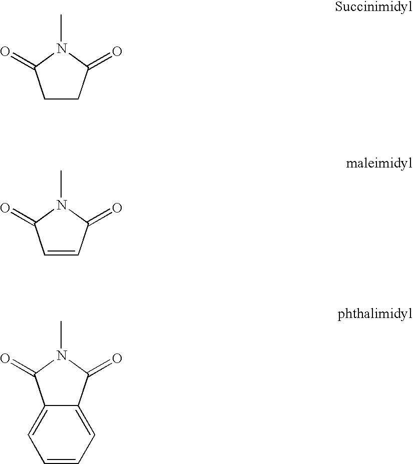

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)NH(CH₃)₂, and —C(═S)NHCH₂CH₃.

Tetrazolyl: a five membered aromatic ring having four nitrogen atoms and one carbon atom,

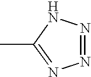

Diazine, including 1,3 diazine, pyrimidine, miazine.

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh.

Examples of cyclic amino groups include, but are not limited to, aziridino, azetidino, piperidino, piperazino, morpholino, and thiomorpholino.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇alkyl group (also referred to as a C₁₋₇alkylthio group), a C₃₋₂₀heterocyclyl group, or a C₅₋₂₀aryl group, preferably a C₁₋₇alkyl group. Examples of C₁₋₇alkylthio groups include, but are not limited to, —SCH₃ and —SCH₂CH₃Sulfonic acid (sulfo): —S(═O)₂OH.

Sulfonate (sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)2OCH₃ and —S(═O)2OCH₂CH₃.

Sulfone (sulfonyl): —S(═O)₂R, wherein R is a sulfone substituent, for example, a C₁₋₇alkyl group, a C₃₋₂₀heterocyclyl group, or a C₅₋₂₀aryl group, preferably a C₁₋₇alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Sulfonyloxy: —OS(═O)₂R, wherein R is a sulfonyloxy substituent, for example, a C₁₋₇alkyl group, a C₃₋₂₀heterocyclyl group, or a C₅₋₂₀aryl group, preferably a C₁₋₇alkyl group. Examples of sulfonyloxy groups include, but are not limited to, —OS(═O)₂CH₃ and —OS(═O)₂CH₂CH₃.

Sulfinyl: —S═O

Sulfinyloxy: —OS(═O)R, wherein R is a sulfinyloxy substituent, for example, a C₁₋₇alkyl group, a C₃₋₂₀heterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfamino: —NR¹S(═O)²OH, wherein R¹ is an amino substituent, as defined for amino groups. Examples of sulfamino groups include, but are not limited to, NHS(═O)²OH and —N(CH³)S(═O)²)H.

Sulfonamino: —NR¹S(═O)²R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇alkyl group, a C₃₋₂₀heterocyclyl group, or a C₅₋₂₀aryl group, preferably a C₁₋₇alkyl group. Examples of sulfinamino groups include, but are not limited to, —NHS(═O)CH3 and —N(CH₃)S(═O)C₆H₅.

Sulfamyl: —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido: —S(═O)₂NR¹R² wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

As mentioned above, a C₁₋₇alkyl group may be substituted with, for example, hydroxy (also referred to as a C₁₋₇hydroxyalkyl group), C₁₋₇alkoxy (also referred to as a C₁₋₇alkoxyalkyl group), amino (also referred to as a C₁₋₇aminoalkyl group), halo (also referred to as a C₁₋₇haloalkyl group), carboxy (also referred to as a C₁₋₇carboxyalkyl group), and C₅₋₂₀aryl (also referred to as a C₅₋₂₀aryl-C₁₋₇alkyl group).

Similarly, a C₅₋₂₀aryl group may be substituted with, for example, hydroxy (also referred to as a C₅₋₂₀hydroxyaryl group), halo (also referred to as a C₅₋₂₀haloaryl group), amino (also referred to as a C₅₋₂₀aminoaryl group, e.g., as in aniline), C₁₋₇alkyl (also referred to as a C₁₋₇alkyl-C₅₋₂₀aryl group, e.g., as in toluene), and C₁₋₇alkoxy (also referred to as a C₁₋₇alkOXY-C₅₋₂₀aryl group, e.g., as in anisole).

These and other specific examples of such substituted groups are also discussed below.

C₁₋₇haloalkyl group: The term “C₁₋₇haloalkyl group,” as used herein, pertains to a C₁₋₇alkyl group in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C₁₋₇perhaloalkyl group.” Examples of C₁₋₇haloalkyl groups include, but are not limited to, —CF₃, —CHF₂, —CH₂F, —CCl₃, —CBr₃, —CH₂CH₂F, —CH₂CHF₂, and —CH₂CF₃.

C₁₋₇hydroxyalkyl: The term “C₁₋₇hydroxyalkyl group,” as used herein, pertains to a C₁₋₇alkyl group in which at least one hydrogen atom has been replaced with a hydroxy group. Examples of C₁₋₇hydroxyalkyl groups include, but are not limited to, —CH₂OH, —CH₂CH₂OH, and —CH(OH)CH₂OH.

C₁₋₇carboxyalkyl: The term “C₁₋₇carboxyalkyl group,” as used herein, pertains to a C₁₋₇alkyl group in which at least one hydrogen atom has been replaced with a carboxy group. Examples of C₁₋₇carboxyalkyl groups include, but are not limited to, —CH₂COOH and —CH₂CH₂COOH.

C₁₋₇aminoalkyl: The term “C₁₋₇aminoalkyl group,” as used herein, pertains to a C₁₋₇alkyl group in which at least one hydrogen atom has been replaced with an amino group. Examples of C₁₋₇aminoalkyl groups include, but are not limited to, —CH₂NH₂, —CH₂CH₂NH₂, and —CH₂CH₂N(CH₃)₂.

C₁₋₇alkyl-C₅₋₂₀aryl: The term “C₁₋₇alkyl-C₅₋₂₀aryl,” as used herein, describes certain C₅₋₂₀aryl groups which have been substituted with a C₁₋₇alkyl group. Examples of such groups include, but are not limited to, tolyl (as in toluene), xylyl (as in xylene), mesityl (as in mesitylene), styryl (as in styrene), and cumenyl (as in cumene).

C₅₋₂₀aryl-C₁₋₇alkyl: The term “C₅₋₂₀aryl-C₁₋₇alkyl,” as used herein, describes certain C₁₋₇alkyl groups which have been substituted with a C₅₋₂₀aryl group. Examples of such groups include, but are not limited to, benzyl (phenylmethyl), tolylmethyl, phenylethyl, and triphenylmethyl (trityl).

C₅₋₂₀haloaryl: The term “C₅₋₂₀haloaryl,” as used herein, describes certain C₅₋₂₀aryl groups which have been substituted with one or more halo groups. Examples of such groups include, but are not limited to, halophenyl (e.g., fluorophenyl, chlorophenyl, bromophenyl, or iodophenyl, whether ortho-, meta-, or para-substituted), dihalophenyl, trihalophenyl, tetrahalophenyl, and pentahalophenyl.

Bidentate Substituents

Some substituents are bidentate, that is, have two points for covalent attachment. For example, a bidentate group may be covalently bound to two different atoms on two different groups, thereby acting as a linker therebetween. Alternatively, a bidentate group may be covalently bound to two different atoms on the same group, thereby forming, together with the two atoms to which it is attached (and any intervening atoms, if present) a cyclic or ring structure. In this way, the bidentate substituent may give rise to a heterocyclic group/compound and/or an aromatic group/compound. Typically, the ring has from 3 to 8 ring atoms, which ring atoms are carbon or heteroatoms (e.g., boron, silicon, nitrogen, phosphorus, oxygen, and sulfur, typically nitrogen, oxygen, and sulfur), and wherein the bonds between said ring atoms are single or double bonds, as permitted by the valencies of the ring atoms. Typically, the bidentate group is covalently bound to vicinal atoms, that is, adjacent atoms, in the parent group.

C₁₋₇alkylene: The term “C₁₋₇alkylene,” as used herein, pertains to a bidentate moiety obtained by removing two hydrogen atoms, either both from the same carbon atom, or one from each of two different carbon atoms, of a C₁₋₇hydrocarbon compound having from 1 to 7 carbon atoms, which may be aliphatic or alicyclic, or a combination thereof, and which may be saturated, partially unsaturated, or fully unsaturated.

Examples of linear saturated C₁₋₇alkylene groups include, but are not limited to, —(CH₂)ₙ— where n is an integer from 1 to 7, for example, —CH₂— (methylene), —CH₂CH₂-(ethylene), —CH₂CH₂CH₂— (propylene), and —CH₂CH₂CH₂CH₂— (butylene).

Examples of branched saturated C₁₋₇alkylene groups include, but are not limited to, —CH(CH₃)—, —CH(CH₃)CH₂—, —CH(CH₃)CH₂CH₂—, —CH(CH₃)CH₂CH₂CH₂—, CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇alkylene groups include, but are not limited to, —CH═CH— (vinylene), —CH═CH—CH2—, —CH═CH—CH₂—CH₂—, —CH═CH—CH₂—CH₂—CH₂—, —CH═CH—CH═CH—, —CH═CH—CH═CH—CH₂—, —CH═CHCH═CH—CH₂—CH₂—, —CH═CH—CH₂—CH═CH—, and —CH═CH—C₁H₂—CH₂-CH═CH—.

Examples of branched partially unsaturated C₁₋₇alkylene groups include, but are not limited to, —C(CH₃)═CH—, —C(CH₃)═CH—CH₂—, and —CH═CH—CH(CH₃)—.

Examples of alicyclic saturated C₁₋₇alkylene groups include, but are not limited to, cyclopentylene (e.g., cyclopent-1,3-ylene), and cyclohexylene (e.g., cyclohex-1,4ylene).

Examples of alicyclic partially unsaturated C₁₋₇alkylene groups include, but are not limited to, cyclopentenylene (e.g., 4-cyclopenten-1,3-ylene), cyclohexenylene (e.g., 2-cyclohexen-1,4-ylene, 3-cyclohexen-1,2-ylene, 2,5-cyclohexadien-1,4-ylene).

C₅₋₂₀arylene: The term “C₅₋₂₀arylene,” as used herein, pertains to a bidentate moiety obtained by removing two hydrogen atoms, one from each of two different ring atoms of a C₅₋₂₀aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboarylene groups,” in which case the group may conveniently be referred to as a “C₅₋₂₀carboarylene” group.

Alternatively, the ring atoms may include one or more heteroatoms, including but not limited to oxygen, nitrogen, and sulfur, as in “heteroarylene groups.” In this case, the group may conveniently be referred to as a “C₅₋₂₀heteroarylene” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀arylene groups which do not have ring heteroatoms (i.e., C₅₋₂₀carboarylene groups) include, but are not limited to, those derived from benzene (i.e., phenyl) (C₆), naphthalene (C₁₀), anthracene (C₁₄), phenanthrene (C₁₄), and pyrene (C₁₆).

Examples of C₅₋₂₀heteroarylene groups include, but are not limited to, C₅heteroarylene groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆heteroarylene groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

C₅₋₂₀Arylene-C₁₋₇alkylene: The term “C₅₋₂₀arylene-C1-7alkylene,” as used herein, pertains to a bidentate moiety comprising a C₅₋₂₀arylene moiety, -Arylene-, linked to a C₁₋₇alkylene moiety, -Alkylene-, that is, -Arylene-Alkylene-.

Examples of C₅₋₂₀arylene-C₁₋₇alkylene groups include, but are not limited to, phenylene-methylene, phenylene-ethylene, phenylene-propylene, and phenylene-ethenylene (also known as phenylene-vinylene).

C₅₋₂₀Alkylene-C₁₋₇arylene: The term “C₅₋₂₀alkylene-C₁₋₇arylene,” as used herein, pertains to a bidentate moiety comprising a C₅₋₂₀alkylene moiety, -Alkylene-, linked to a C₁₋₇arylene moiety, -Arylene-, that is, -Alkylene-Arylene-.

Examples of C₅₋₂₀alkylene-C₁₋₇arylene groups include, but are not limited to, methylene-phenylene, ethylene-phenylene, propylene-phenylene, and ethenylene-phenylene (also known as vinylene-phenylene).

Included in the above are the well known ionic, salt, solvate (e.g., hydrate), and protected forms of these substituents. For example, a reference to carboxylic acid (—COOH) also includes carboxylate (—COO⁻). Similarly, a reference to an amino group includes a salt, for example, a hydrochloride salt, of the amino group. A reference to a hydroxyl group also includes conventional protected forms of a hydroxyl group. Similarly, a reference to an amino group also includes conventional protected forms of an amino group.

In particularly preferred embodiments of the invention, the term ‘substituents’ may include but is not limited to the group consisting of hydrogen, C₂-C₂₀ alkenyl, C₂-C₂₀ alkynyl, C₁-C₂₀ alkyl, aryl, C₁-C₂₀ carboxylate, C₁-C₂₀ alkoxy, C₂-C₂₀ alkenyloxy, aryloxy, C₂-C₂₀ alkoxycarbonyl, C₁-C₂₀ alkylthio, C₁-C₂₀ alkylsulfonyl, or C₁-C₂₀ alkylsulfinyl; each optionally substituted with C₁-C₅ alkyl, halogen, C₁-C₅ alkoxy, or with a phenyl group optionally substituted with halogen, C₁-C₅ alkyl, or C₁-C₅ alkoxy, and acetylene comprising from 2 to 20 carbon atoms. In specific embodiments acetylene substituents may be particularly preferred. In other preferred embodiments, each substituent may be a metallocycles or heterocyclicmetallocycles (to include porphyrins and phthalocyaines), or perfluoroalkyl or diazine, attached either directly to the linear series of five fused carbon rings, or attached via acetylene. Each substituent is selected independently to other substituents unless otherwise indicated.

Ortho-Quinodimethanes: refer, at least in preferred embodiments, to unstable dienes that may be trapped readily in the presence of either acylic or cyclic dienophiles. Typical precursors are illustrated in Scheme 2 (see later). The utility of the tetra-bromide precursor is the sequential aromatization of the B and D rings in the same reaction vessel once the double cycloaddition complete, to afford the pentacene quinone directly.

The tricyclic ring system illustrated below may be named by established precedent as: Tetrakisnaphthoperhydro[0.0]paracyclophane or Tetrakisnaphthotricyclo[4.2.2.2^{2,5}]dodecane or Tetrakisnaphtnotricyclo[6.2.2.0^{5,10}]dodecane

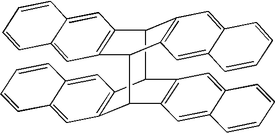

### DETAILED DESCRIPTION OF THE INVENTION

Functionalized pentacene compounds with substituents on the terminal A and E rings are predicted to have better intermolecular π-stacking than compounds with substituents attached to the central C ring. However, few synthetic routes to pentacenes with substituents on the A and E rings are currently known. Pentacene has a greater electron density and reactivity at the central C ring (Schleyer P. R. et al. Org Lett (2001) 3, 3646; Randić M. Chem Rev (2003) 103, 3449) making selective functionalization of pentacene on the A and E rings difficult.

In preferred embodiments, the inventors of the present invention have developed novel pathways for the production of compounds comprising a linear series of five fused carbon rings, which are believed to present significant advantages over the methods of the prior art. Without wishing to be bound by theory, the methods of the present invention present the opportunity to manufacture, at least in preferred embodiments, alternative pentacene substitutions at the A and E rings, thereby providing a greater degree of substituent flexibility. Such substituents can be used to more carefully tune the electronic properties and/or affect the solid-state packing of the pentacene derivatives for use in electronic components such as thin-film transistors. However, the invention is not limited in this regard. The methods of the present invention permit the formation of a wide range of compounds with a core structure comprising a linear series of five fused carbon rings. The novel methods allow facile access to a wide range of compounds including substituted pentacenes and ortho-quinodimethanes that were previously unobtainable or difficult to obtain.

Such compounds include those of formula III or IV: 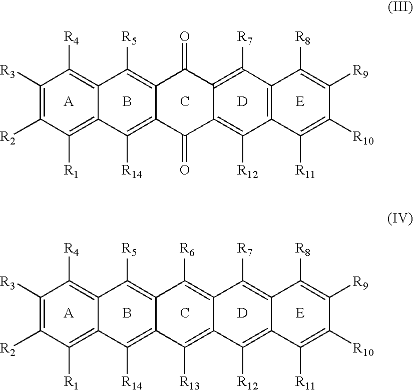
wherein R₁ to R₁₄ are each independently unsubstituted or substituted. The methods, at least in preferred embodiments, allow access to compounds comprising at least one substituent on each of the A and E rings of the core structure, or compounds comprising at least one substituent on each of the A and E rings, and at least one substituent on at least one of the B, C, or D rings of the core structure. The methods also allow access to compounds with substitutions at the 2, and the 9 or 10 positions, and other positions in the five fused carbon ring system. In most preferred embodiments, the methods of the present invention permit the production of compounds comprising a linear series of five fused carbon rings substituted with acetylene groups, or by substituents that are attached to the core structure via a linker comprising one or more triple bonds. Such compounds are amenable to further substitution or coupling via the acetylene moieties.

In one particularly preferred embodiment of the present invention there is provided a method for the preparation of an compound comprising at least one linear series of five fused carbon rings, each carbon ring being saturated, unsaturated, or aromatic, and being unsubstituted or substituted, the method comprising the steps of:
(a) providing an unsubstituted or substituted benzoquinone;
(b) providing an unsubstituted or substituted acyclic, cyclic, heterocyclic or ortho-quinodimethane diene;
(c) performing a double or stepwise cycloaddition reaction between the benzoquinone and the diene compound to generate a core structure comprising five fused carbon rings;
(d) optionally performing a ring opening reaction to covert a bridged form of each of rings B and D, if present, to an unbridged form;
(e) optionally performing an aromatization reaction or equivalent on the B, and D rings of the core structure;
(f) optionally replacing or adding selected substituents;
(g) optionally subjecting the compound to reducing conditions to generate a corresponding unsubstituted or substituted pentacene;
(h) optionally separating isomeric products; and
(i) optionally performing a coupling reaction to link two or more core structures;
   wherein any one or more of optional steps (d), (e), (f), (g), (h), and (i) may be conducted, and any two or more of steps (d), (e), (f), (g), (h) and (i) may be performed in any order.

In preferred embodiments the benzoquinone has the general formula I: 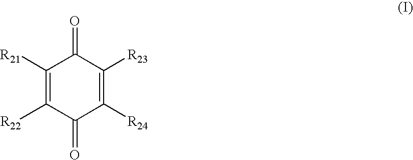
wherein each R group is independently selected from hydrogen and the group consisting of an electron-withdrawing group, halogen, and a protonated amine. Moreover, the diene compound preferably has the general formula IIa or IIb: 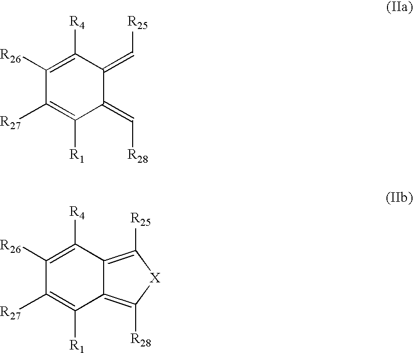
wherein each R group is H or any group that does not interfere with the capacity of the diene to undergo a cycloaddition reaction with benzoquinone, and X is C, O, S, or N. Most preferably the reaction comprises a double Diels-Alder reaction between the benzoquinone and two diene molecules. In most preferred embodiment, R₂₅ may be considered a leaving group. For example, R₂₅ may comprise OAlk wherein each Alk comprises an alkyl group of from 1 to 12 carbon atoms, or R₂₅ and R₂₈ may be halogen.

In specific embodiments of the invention, dienes of the formula IIb may result in the production of linear five-fused carbon ring structures after ring opening and aromatization.

The methods defined above are within the scope of the present invention, and present further opportunities for selective substituent addition to the resulting core structure of five fused carbon rings.

The methods of the present invention are specifically designed, at least in preferred embodiments, for the production of pentacene compounds with substitutions in the 2 and 9 or 10 positions. Such pentacene compounds are particularly suited for use in electronic applications by virtue of their desirable crystal packing properties (see later). For this reason, the diene compounds of formula (IIa) or (IIb) preferably comprise substituents at R₂₆ or R₂₇, each comprising A-B, wherein A is a protective group, and B is a group to be protected. In this way, the methods of the invention may generate compounds of the formula III: 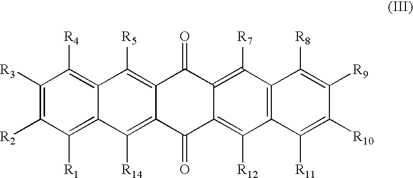
wherein R₁ to R₁₄ are each independently unsubstituted or substituted, wherein preferably at least R₂ and R₉ or R₁₀ are substituted with A-B, or an alternative substituent. In this case, the substituents at R, and at R₉ or R₁₀ are derived from R₂₆ or R₂₇ of the diene substrates.

Moreover, optional reduction of the compound of formula III can lead to the production of pentacene compounds of formula IV: 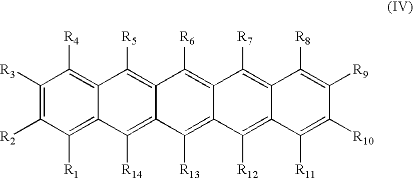
wherein preferably, R₁ to R₁₄ are each independently unsubstituted or substituted, and wherein more preferably at least R₂ and R₉ or R₁₀ are substituted with A-B, or an alternative substituent. The substituents at the R₂ and R₉ or R₁₀ positions are preferably selected from acetylene, alkyl, aryl, heteroaryl, alkenyl, and alkynyl. Most preferably, R₂ and R₉ or R₁₀ may comprise acetylene or a linker comprising one or more triple bonds, optionally substituted by halogen. Preferably, each A-B is a a silica-based protective group. For example, each A may comprise a silyl ether such as TMS, TES, TBS, and TIPS, and each B may be O, S, Se, or N.

In another preferred embodiment, the method of the present invention may comprise step (i) as recited above, thereby to generate an oligomeric compound comprising pentacyclic units linked by acetylene groups at the 2 and 9 or 10 positions. Without wishing to be bound by theory, it is considered that many such oligomeric chains of core structures (each core structure comprising a linear array of five fused carbon rings) may exhibit very desirable crystal packing and electronic properties by virtue of optimal ε-orbital electron overlap. The present invention therefore encompasses oligomeric or polymeric forms of the compounds disclosed herein.

In most preferred embodiments, the methods of the present invention are for the preparation of pentacenes at least comprising substitutions at the 2, and the 9 or 10 positions, the method comprising the steps of:
(a) performing a stepwise or double Diels-Alder reaction by reacting a compound of formula IIa or IIb: 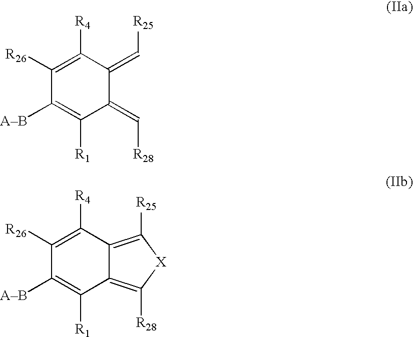
   wherein A is a protective group, B is a group to be protected, each R group is independently selected from H or a substituent, and X is C, O, S, or N, with a compound of formula II: 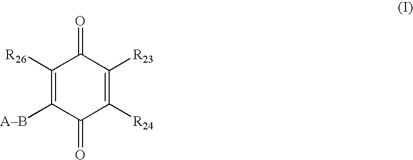
   wherein each R group is independent selected from H or a substituent to form a mixture of compounds of formula V and VI: 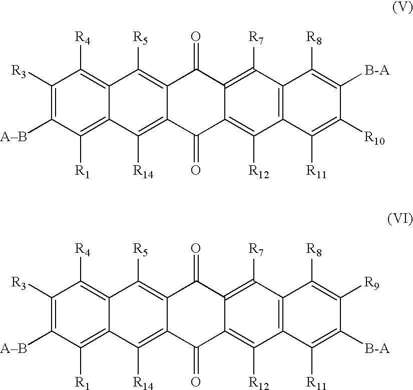
wherein A is a protective group, B is a group to be protected, and each R group is independent selected from H or a substituent;
(b) optionally separating the compounds of formula (V) and formula (VI), and selecting the compound of formula (V) and/or the compound of formula (VI) for further processing;
(c) replacing each A or each A-B with any substituent, with or without a linker comprising one or more triple bonds to form a 2,9- and/or a 2,10-disubstituted quinone;
(d) subjecting the 2,9- and/or the 2,10-disubstituted quinone to reducing dehydrating conditions to generate a pentacene substituted at least in the 2 position, and the 9 or 10 position.

The step of optional separation of the isomers V and VI may involve, for example, high performance liquid chromatography, fractional crystallization, or other suitable techniques that are well known in the art.

It should be noted that the diene may preferably include a protective group that will ultimately confer functionalization to the A and/or E ring of the pentacene. Any protective group may be used for this purpose in accordance with the corresponding protected group, and the protective group may be substituted as desired at a later stage. Particularly preferred protective groups include silyl ethers, which may be selected from, but not limited to, TMS, TES, TBS, or TIPS. Such protective groups can be substituted by methods known in the art. For example, monoquinone compounds having only silyl ether substituents at the 2, and the 9 or 10 positions (originating from R₂₇ of the diene) may be subjected to desilylation and triflation to generate the compounds shown in formulae (V) and (VI):

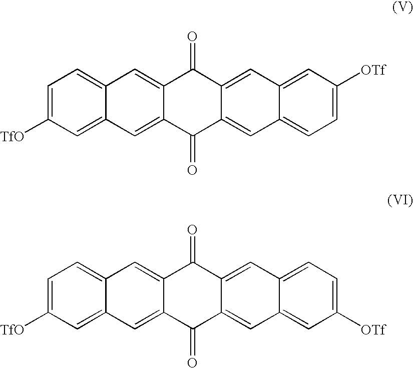

Further processing of the compounds of formula VII or VIII can be carried out, for example by coupling reactions, such as for example a Sonogashira reaction involving Pd-coupling. Subsequent reduction of the monoquinone core can generate the corresponding disubstituted pentacene.

The methods of the present invention have proven highly successful and flexible in the production of 2,9- and 2,10-disubstituted pentacene compounds. Importantly, the methods of the present invention present opportunities for the production of novel 2,9- or 2,10-disubstituted pentacenes comprising acetylene substituents, which are themselves very useful as intermediates for the generation of alternative substitutions or for coupling reactions.

The present invention further encompasses a wide range of compounds that at least comprise a linear series of five fused carbon rings.

Such compounds include those of the formula III: 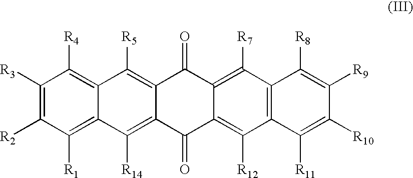
wherein R₁ to R₁₄ are each independently unsubstituted or substituted.

In preferred embodiments, the present invention provides for a compound of formula IV: 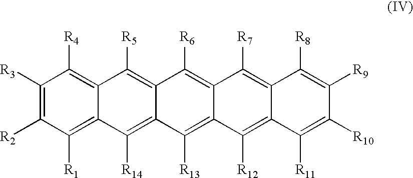
wherein R₁ to R₁₄ are each independently unsubstituted or substituted.

Preferably, the compounds of formula IV include the proviso that the compounds of formula IV exclude pentacenes comprising only alkyl groups at R₂ and R₉ and/or R₁₀.

Preferably, the compounds of formula IV include the proviso that when at least one of R₁, R₂, R₃, R₄, R₈, R₉, R₁₀, and R₁₁ are substituted with an electron-donating substituent, or a halogen, then the compound must include at least one further substituent at R₅, R₆, R₇, R₁₂, R₁₃, or R₁₄.

Preferably, the compounds of formula III or IV comprise at least one substituent on each of the A and E rings of the core structure. More preferably, the compound comprises at least one substituent on each of the A and E rings, and at least one substituent on at least one of the B, C, or D rings of the core structure. More preferably, the compound comprises substituents at least at the 2, and the 9 or 10 positions. Most preferably, the substituents at the 2, and the 9 or 10 positions are acetylene groups, or are each attached to the core structure via a linker comprising one or more triple bonds. Preferably, in accordance with the compound of formula III each substituent is independently selected from hydroxyl, alkyl, alkoxy, alkenyl, alkynyl, aryl, heteroaryl, acetylene, halogen, and triflate. More preferably, each substituent is substituted by alkyl or halogen.

In selected embodiments, the methods of the invention provide for rapid synthesis of the compounds of the invention. Importantly, the methods afford a significant degree of flexibility with regard to the substituents located on the substrates during synthesis of the five fused carbon ring core structure. Moreover, the possibility of using cyclic dienes to generate bicyclo compounds presents a further opportunity to manipulate the substituents on the core structure. The optional reduction of the quinone compounds of the invention presents further opportunities for substituent addition or replacement.

The pentacene compounds of the present invention are differentiated over those of the prior art by virtue of the wide range of possible substituents that can be positioned on the A and E rings, as well as the B, C, and D rings, and also their solubility in organic solvents. Their solubility is such that in selected embodiments the compounds of the present invention may be useful for ink jet fabrication. In one particularly advantageous embodiment, the A and E rings may comprise acetylene substituents, or may comprise substituents attached to the core structure via a linker of one or more triple bonds. This option presents unique opportunities for the provision of a wide range of substituents at such positions on the core structure, for example by manipulation or replacement of the acetylene. In further selected embodiments, the pentacene compounds of the invention may include Buckminsterfullerene-containing substituents and/or phthalocyanine substituents to generate pentacene compounds suitable for use, for example, in solar cells or components thereof.

Generation of Organic Thin Film Transitors (OTFTs) or Other Electronic Components

The present invention provides methods for the production of compounds suitable for use in the manufacture of components, specifically organic semiconductor components, of Organic Thin Film Transistors and other electronic devices. The present invention encompasses such components, their manufacture, and OTFTs containing them. The methods of the present invention may be useful in the production of any types of OTFTs that incorporate pentacene derivative molecules.

Typically, a thin film transistor includes a gate electrode, a gate dielectric on the gate electrode, a source electrode and a drain electrode adjacent to the gate dielectric, and a semiconductor layer adjacent to the gate dielectric and adjacent to the source and drain electrodes. More specifically, an organic thin film transistor (OTFT) has an organic semiconductor layer. Such OTFTs are known in the art as shown, for example, in U.S. Pat. No. 6,433,359, issued Aug. 13, 2002, and U.S. Pat. No. 6,617,609 issued Sep. 9, 2003, which are herein incorporated by reference.

A substrate can be used to support the OTFT, e.g., during manufacturing, testing, storage, use, or any combination thereof. The gate electrode and/or gate dielectric may provide sufficient support for the intended use of the resultant OTFT and another substrate is not required. For example, doped silicon can function as the gate electrode and support the OTFT. In another example, one substrate may be selected for testing or screening various embodiments while another substrate is selected for commercial embodiments. In another embodiment, a support may be detachably adhered or mechanically affixed to a substrate, such as when the support is desired for a temporary purpose. For example, a flexible oligomeric substrate may be adhered to a rigid glass support, which support could be removed. In some embodiments, the substrate does not provide any necessary electrical function for the OTFT. This type of substrate is termed a “non-participating substrate” in this document.

Useful substrate materials can include organic and/or inorganic materials. For example, the substrate may comprise inorganic glasses, ceramic foils, polymeric materials, filled polymeric materials, coated metallic foils, acrylics, epoxies, polyamides, polycarbonates, polyimides, polyketones, poly(oxy-1,4-phenyleneoxy-1,4-phenylenecarbonyl-1,4-phenylene) (sometimes referred to as poly(ether ether ketone) or PEEK), polynorbornenes, polyphenyleneoxides, poly(ethylene naphthalenedicarboxylate) (PEN), poly(ethylene terephthalate) (PET), poly(phenylene sulfide) (PPS), and fiber-reinforced plastics (FRP).

The gate electrode can be any useful conductive material. For example, the gate electrode may comprise doped silicon, or a metal, such as aluminum, chromium, copper, gold, silver, nickel, palladium, platinum, tantalum, and titanium. Conductive polymers also can be used, for example polyaniline, poly(3,4-ethylenedioxythiophene)/poly(styrene sulfonate) (PEDOT:PSS). In addition, alloys, combinations, and multilayers of these materials may be useful.

The gate dielectric is provided on the gate electrode, for example, through a deposition method. This gate dielectric electrically insulates the gate electrode under the operating conditions of the OTFT device from the balance of the device. Thus, the gate dielectric comprises an electrically insulating material. The gate dielectric should have a dielectric constant above about 2, more preferably above about 5. The dielectric constant of the gate dielectric also can be very high, for example, 80 to 100 or even higher. Useful materials for the gate dielectric may comprise, for example, an organic or inorganic electrically insulating material, or combinations thereof.

The gate dielectric may comprise a polymeric material, such as polyvinylidenefluoride (PVDF), cyanocelluloses, polyimides, epoxies, etc. In some embodiments, an inorganic capping layer comprises the outer layer of an otherwise polymeric gate dielectric for improved bonding to the polymeric layer and/or improved dielectric properties.

Specific examples of inorganic materials useful for the gate dielectric include strontiates, tantalates, titanates, zirconates, aluminum oxides, silicon oxides, tantalum oxides, titanium oxides, silicon nitrides, barium titanate, barium strontium titanate, barium zirconate titanate, zinc selenide, and zinc sulfide. In addition, alloys, combinations, and multilayers of these can be used for the gate dielectric. Of these materials, aluminum oxides, silicon oxides, silicon nitrides, and zinc selenide are preferred.

The gate dielectric can be deposited in the OTFT as a separate layer, or formed on the gate such as by oxidizing, including anodizing, the gate material to form the gate dielectric.

The source electrode and drain electrode are separated from the gate electrode by the gate dielectric, while the organic semiconductor layer can be over or under the source electrode and drain electrode. The source and drain electrodes can be any useful conductive material. Useful materials include those materials described above for the gate electrode, for example, aluminum, barium, calcium, chromium, copper, gold, silver, nickel, palladium, platinum, titanium, polyaniline, PEDOT:PSS, other conducting polymers, alloys thereof, combinations thereof, and multilayers thereof.

The thin film electrodes (e.g., gate electrode, source electrode, and drain electrode) can be provided by any useful means such as physical vapor deposition (e.g., thermal evaporation, sputtering), plating, or ink jet printing. The patterning of these electrodes can be accomplished by known methods such as shadow masking, additive photolithography, subtractive photolithography, printing, transfer printing, microcontact printing, and pattern coating.

The organic semiconductor layer, produced in accordance with the present invention, can be provided by any useful means, such as for example, vapor deposition, solution deposition, spin coating, and printing techniques, all of which are well known in the art.

Importantly, the compounds of the present invention can be used in the manufacture of a wide range of electronic devices and semiconductor components, including but not limited to, Organic Thin Film Semiconductor (OTFS), an Organic Field-Effect Transistor (OFET), an Organic Light Emitting Diode (OLED), a solar cell, and a device for solar energy conversion.

The following schemes illustrate examples methods for the production of substrates, intermediates, or products of the methods of the invention, or illustrate examples of compounds of the invention. The schemes are for illustrative purposes only and are in no way intended to limit the scope of the invention described herein or as defined in the appended claims.

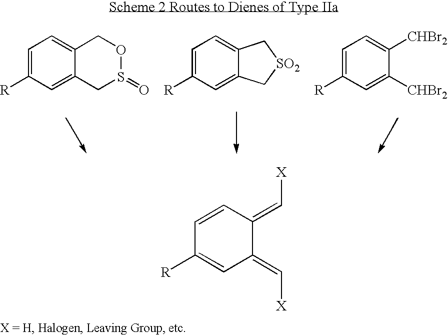

An alternative cycloaddition approach is to use a diene of type IIb and cleave the oxygen bridge to either isolate the intermediates or generate the B and D aromatic rings directly.

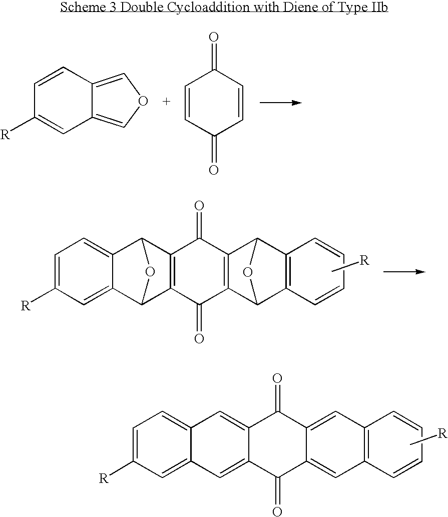

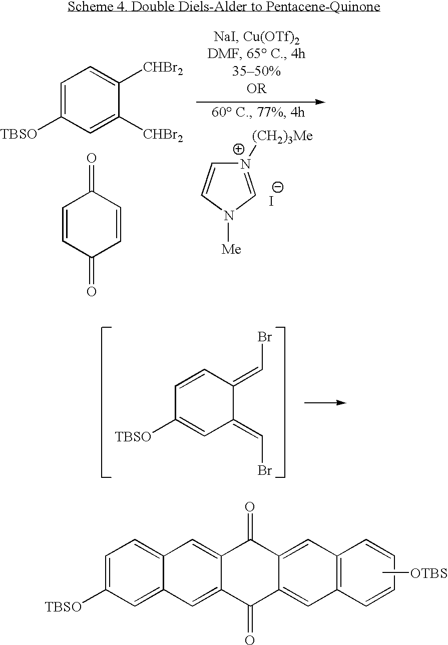

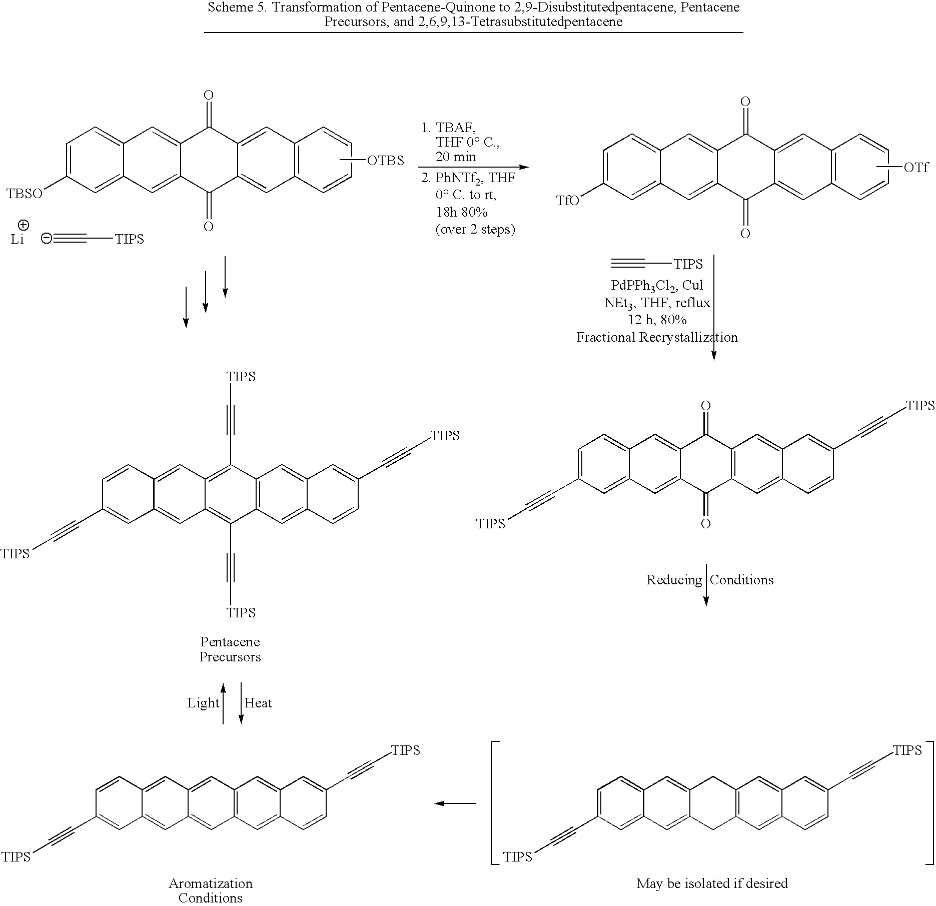

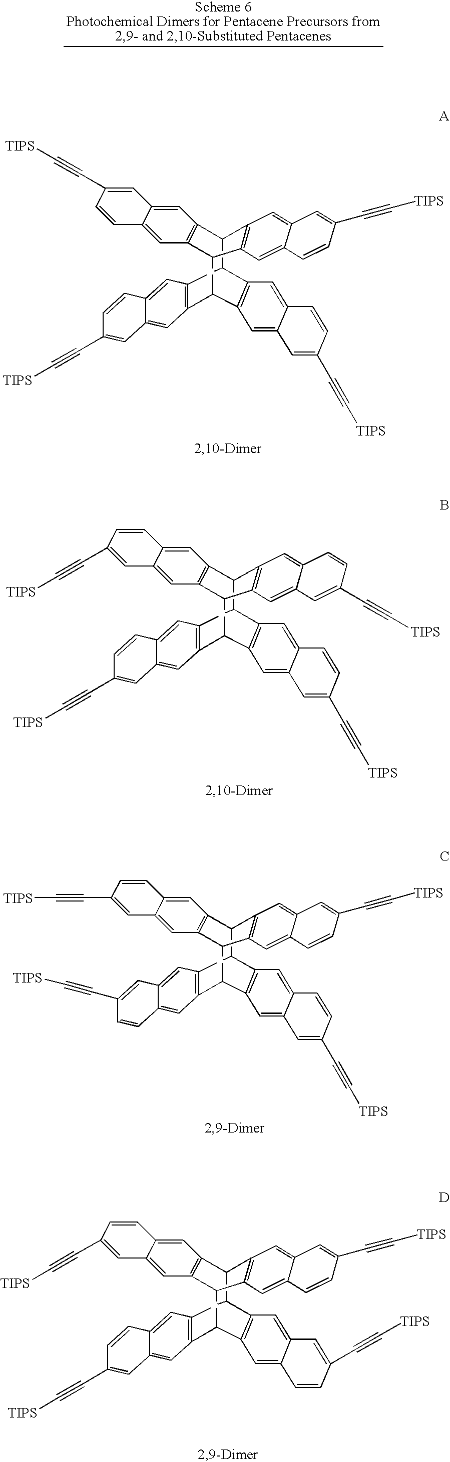

### EXAMPLES

### Example 1a

### Synthesis of Benzoquinone Adducts

Benzoquinone (0.040 g, 0.368 mmol) was dissolved in dry dimethylformamide (20 mL) and (3,4-bis(dibromomethyl)phenoxy)tert-butyl)dimethylsilane (0.407 g, 0.736 mmol, 2 eq.) was added, followed by Cu(OTf)₂ (0.013 g, 0.037 mmol, 0.1 eq.) and NaI (0.717 g, 4.786 mmol, 13 eq.), respectively. The reaction was stirred at 65° C. for 8 hours. Once the absence of initial benzoquinone aliquot was confirmed by TLC, further aliquots of benzoquinone were added (0.010 g, 0.093 mmol, 0.25 eq., three further additions over the duration of reaction) until complete consumption of (3,4-bis(dibromomethyl)phenoxy)(tert-butyl)dimethylsilane was observed. Reaction was cooled to room temperature (22° C.) and the solution turned from brown to yellow upon the addition of cold sat. Na₂S₂O₃. The yellow precipitate that formed was filtered and washed with H₂O, then taken up with CH₂Cl₂ and concentrated. The impurities were dissolved in acetone and the product was filtered through a sintered glass funnel, which provided 2,9 and 2,10-bis-(tert-butyl-dimethylsilyloxy)-pentacene-6,13-dione as a yellow solid (0.106 g, 50% (30%-50%)).

### Example 1b

### Synthesis of Benzoquinone Adducts

3,4-Bis(dibromomethyl)phenoxy)tert-butyl)dimethylsilane (1.10 g, 2 mmol) and benzoquinone (218 mg, 2 mmol) were added to ionic liquid 1-butyl-3-methylimidazolim iodide (5 g) under stirring. The mixture was heated to 60° C. for 2 h. The mixture was then washed with ether (4×), and the upper ether layer was decanted and combined. The ether was removed under reduced pressure and the solid was washed with acetone to afford 2.9 and 2,10-bis-(tert-butyl-dimethylsilyloxy)-pentacene-6,13-dione as a yellow solid (436.6 mg, 77%) The remaining ionic liquid phase was dried under vacuum and directly reused in the subsequent runs.

2,9 isomer: mp: >270° C.; IR (solution cell: CH₂Cl₂): ν=3055, 3005, 1712, 1431, 1265, 1258, 1222, 909, 767, 756, 748, 729; ¹H NMR (300 MHz, CDCl₃) δ 8.83 (s, 2H), 8.73 (s, 2H), 7.98 (d, J=9 Hz, 2H), 7.41 (d, J=2.1 Hz, 2H), 7.27 (d, J=2.4 Hz, 1H), 1.02, (s, 9H), 0.29 (s, 6H); ¹³C NMR (75 MHz, CDCl₃) δ 183.5 (C), 157.1 (C), 137.4 (C), 132.2 (CH), 131.4 (C), 131.2 (C), 130.0 (CH), 129.3 (C), 128.4 (CH), 126.2 (CH), 116.9 (CH), 26.0 (CH₃), 18.7 (C), −3.9 (CH₃); MS (EI) m/z 568 (M⁺) (63), 545 (4.6), 511 (100), 455 (14), 227 (28); HRMS calculated for (M⁺) 568.24651, found 568.24652.

### Example 2

### Synthesis of Bistriflates

2,9 and 2,10-Bis-(tert-butyl-dimethylsilyloxy)-pentacene-6,13-dione (0.177 g, 0.312 mmol) were dissolved in THF (100 mL) and cooled to 0° C. A solution of tert-butylammonium fluoride in THF (1.0 M, 0.69 mL, 0.686 mmol, 2.2 eq.) was added and the reaction turned from yellow to deep blue. After 15 min., Tf₂NPh (0.334 g, 0.936 mmol, 3 eq.) dissolved in THF (5 mL) was cannulated into the reaction flask and then warmed to rt (22° C.). The reaction turned from deep blue to red to yellow. After 24 h, the reaction was concentrated to 50 mL, diluted with ether, washed with 10% HCl, 5% NaHCO₃ and H₂O. Then it was concentrated to 20 mL and filtered through a sintered glass funnel to obtain the 2,9 and 2,10-Bis(trifluoromethylsulfonyl)pentacene-6,13-dione compounds (0.150 g, 80%) and used directly in the next step

### Example 3

### Synthesis of Triisopropylsilyl-Acetylenic Pentacenes

The mixture of 2,9 and 2,10-bis-(trifluoromethylsulfonyl)pentacene-6,13-diones (0.070 g, 0.116 mmol) were dissolved in THF (20 mL), followed by CuI (0.004 g, 0.023 mmol, 0.2 eq.), Pd(PPh₃)Cl₂ (0.008 g, 0.012 mmol, 0.1 eq.) and NEt₃ (2 mL). After degassing, TIPS-acetylene (65 μL, 0.290 mmol, 2.5 eq.) was added and heated at reflux for 12 h. The reaction was cooled to rt and filtered through a pad of silica (95:5, PE/EA) which afforded the 2,9- and 2,10-bis[(triisopropylsilyl)ethynyl]pentacene-6,13-dione compounds (0.057 g, 66%). The isomers were separated by fractional recrystallization to give the 2,9 isomer as yellow needles and the 2,10 isomer as a pale yellow powder. 2,9 isomer: mp: >270° C.; IR (CH₂Cl₂): ν=3058, 2956, 2929, 2864, 2150, 1713, 1675, 1614, 1454, 1310, 1192, 990; ¹H NMR (300 MHz, CDCl₃) δ 8.85 (s, 4H), 8.21 (s, 2H), 8.01 (d, J=8.3, 2H), 7.69 (d, J=7.8 Hz, 2H), 1.16 (s, 42H); ¹³C NMR (75 MHz, CDCl₃) δ 182.9 (C), 135.2 (C), 134.8 (C), 133.9 (CH), 132.7 (CH), 131.5 (C), 131.2 (C), 130.3 (CH), 129.9 (CH), 129.8 (CH), 125.2 (C), 106.7 (C), 95.1 (C), 19.1 (CH₃), 11.7 (CH); MS (EI) m/z 668 (M⁺) (2), 625 (28), 555 (8), 162 (22), 69 (100); HRMS calculated for (M⁺) 668.35058, found 668.35059. 2,10 isomer: mp: >270° C.; IR (CH₂Cl₂): ν=3054, 2948, 2933, 2868, 2154, 1678, 1614, 1454, 1390, 1177, 994, 827; ¹H NMR (300 MHz, CDCl₃) δ 8.84 (s, 4H), 8.20 (s, 2H), 8.00 (d, J=8.4 Hz, 2H), 7.68 (dd, J=8.4 and 1.5 Hz, 2H), 1.16 (s, 42H); ¹³C NMR (75 MHz, CDCl₃) δ 182.9 (C), 182.9 (C), 135.2 (C), 134.8 (C), 133.9 (CH), 132.7 (CH), 131.4 (C), 131.2 (C), 130.3 (CH), 129.8 (CH), 129.8 (CH), 125.1 (C), 106.6 (C), 95.1 (C), 19.1 (CH₃), 11.7 (CH); MS (EI) m/z 668 (M⁺) (9), 625 (100), 583 (23), 555 (34), 419 (5), 162 (12), 70 (15); HRMS calculated for (M⁺) 668.35058, found 668.35059.

### Example 4

### Reduction-Aromatizations to Pentacene and Generation of Pentacene Precursors

2,10-Bis[(triisopropylsilyl)ethynyl]pentacene-6,13-dione (0.025 g, 0.038 mmol) was dissolved in THF (10 mL) and degassed. At 0° C., AlCl₃ (0.051 g, 0.379 mmol, 10 eq.) and LiAlH₄ (0.007 g, 0.189 mmol, 5 eq.) were added and the reaction was heated at reflux for 15 h. The reaction was cooled to RT and ethyl acetate was added to quench the excess LiAlH₄. The salts were precipitated and filtered after dropwise addition of saturated aqueous. NaCl. The organic layer was dried, filtered and concentrated. Recrystallization in petroleum ether afforded the 2,10-bis(triisopropylsilylethynyl)-6,13-dihydropentacene as a white solid (0.023 g, 95%). mp: 73-5° C.; IR (thin-film): ν=2942, 2891, 2864, 2152, 1675, 1462, 1229, 1072, 882, 697; ¹H NMR (300 MHz, CDCl₃) δ 7.93 (s, 1H), 7.69 (m, 6H), 7.45 (d, J=8.3 Hz, 2H), 4.19 (s, 4H), 1.15 (s, 42H); ¹³C NMR (75 MHz, CDCl₃) δ 136.9 (C), 136.7 (C), 132.3 (C), 132.2 (C), 131.6 (CH), 128.8 (CH), 127.5 (CH), 125.5 (CH), 125.4 (CH), 120.7 (C), 108.0 (C), 91.0 (C), 37.8 (CH₂), 37.7 (CH₂), 19.1 (CH₃), 11.7 (CH); MS (EI) m/z 640 (M⁺) (42), 597 (100), 555 (22), 415 (6), 277 (8), 214 (25); HRMS calculated for (M⁺) 640.39205, found 640.39206.

2,9-Bis(triisopropylsilylethynyl)-6,13-dihydropentacene: ¹H NMR (500 MHz, CDCl₃) δ 7.94 (br. s, 2H), 7.70 (d, J=8.4 Hz, 2H), 7.69 (s, 2H), 7.66 (s, 2H), 7.47 (dd, J=8.4, 1.4 Hz, 2H), 4.16 (s, 4H), 1.18 (s, 42H); ¹³C NMR (125 MHz, CDCl₃) δ 136.9, 136.6, 132.3, 132.2, 131.7, 128.8, 127.6, 125.5, 125.3, 120.7, 108.1, 91.0, 37.7, 19.2, 11.8; MS (m/z, EI) 638 (M+-2H).

Alternate Method: A mixture of aluminum (168 mg), carbon tetrabromide (16.8 mg) and mercuric chloride (3.4 mg) in dry cyclohexanol (2.5 mL) were heated at reflux for 1.5 hours under argon in a Schlenk tube. A dark grey mixture was generated and the reaction was cooled to room temperature. A sample of either 2,9- or 2,10-bis(triisopropylsilylethynyl)-5,7,12,14-pentacenediquinone (100 mg, 0.143 mmol) was added to the reaction and the mixture was refluxed for further 2 hours to afford a purple solution. The reaction flask was then cooled to room temperature. Hexane (10 mL) was degassed 4 times and added to the reaction mixture via a syringe to wash the dark grey residue. The mixture was transferred to a round-bottom-flask and both hexanes and cyclohexanol were distilled under vacuum to afford a dark purple residue containing the requisite pentacene. Exposure to light affords a mixture of the two regioisomeric dimers reflecting the 2,9- or 2,10-substitutent pattern that were separated by chromatography (hexane).

2,10-Dimer A: White solid. mp: 190-192° C.; ¹H NMR (500 MHz, CDCl₃) δ 7.66 (s, 4H), 7.41 (d, J=8.5 Hz, 4H), 7.38 (s, 4H), 7.36 (s, 4H), 7.23 (dd, J=8.5, 1.0 Hz, 4H), 5.08 (d, J=6.1 Hz, 4H), 1.09 (s, 84H); ¹³C NMR (125 MHz, CDCl₃) δ 140.8, 140.6, 131.4, 131.2, 128.4, 127.0, 125.4, 125.3, 120.1, 107.4, 90.4, 53.8, 53.6, 18.6, 11.2; MS (m/z, ES) 1316 (M+K⁺).

2,10-Dimer B: White solid. mp: 208-210° C.; ¹H NMR (500 MHz, CDCl₃) δ 7.65 (s, 4H), 7.42 (d, J=8.3 Hz, 4H), 7.38 (s, 4H), 7.37 (s, 4H), 7.23 (dd, J=8.3, 1.5 Hz, 4H), 5.08 (s, 4H), 1.08 (s, 84H); ¹³C NMR (125 MHz, CDCl₃) δ 140.9, 140.6, 131.4, 131.1, 128.4, 127.0, 125.4, 125.3, 120.1, 107.4, 90.3, 53.7, 53.6, 18.6, 11.2; MS (m/z, ES) 1316 (M+K⁺).

2,9-Dimer C: White solid. mp: 208-210° C.*; ¹H NMR (500 MHz, CDCl₃) δ 7.65 (s, 4H), 7.42 (d, J=8.7 Hz, 4H), 7.39 (s, 4H), 7.35 (s, 4H), 7.23 (dd, J=8.7, 1.5 Hz, 4H), 5.08 (s, 4H), 1.08 (s, 84H); MS (m/z, ES) 1316 (M+K⁺).

2,9-Dimer D: White solid. mp: 208-210° C.*; ¹H NMR (500 MHz, CDCl₃) δ 7.64 (s, 4H), 7.43 (d, J=8.5 Hz, 4H), 7.38 (s, 4H), 7.36 (s, 4H), 7.23 (dd, J=8.5, 1.6 Hz, 4H), 5.08 (s, 4H), 1.08 (s, 84H); MS (m/z, ES) 1316 (M+K⁺).

* The 2,9-photochemical dimers are white solids that begin to turn purple at ˜150° C. and turn completely purple by ˜170° C. At this temperature they are still solids and do not melt fully until 208-210° C. Based on the unique purple colour of pentacene it may be concluded that the photochemical dimer sublimes into two molecules of the pentacene at ˜170° C. rather than melting. Thus during sublimation the dimers dissociate to form two new identical molecules of pentacene. The melting point observed at 208-210° C. is thus the melting point of the disubstituted pentacene generated at 170° C. At present this is the best way to obtain the very pure samples of these pentacenes required for thin film electronic applications.

Alternative Method: In separate flasks, 2,10-bis(2-(triisopropylsily)ethynyl)-6,13-dihydropentacene (0.023 g, 0.036 mmol) and DDQ (0.0172 g, 0.076 mmol, 2.1 eq.) were dissolved in benzene (7.5 mL each) and degassed. The solution of DDQ was cannulated into the solution of 2,10-bis(2-(triisopropylsily)ethynyl)-6,13-dihydropentacene and heated at reflux. After 1 hour, the solution was cooled to rt and concentrated. Purification by column chromatography (9:1; petroleum ether/ethyl acetate) afforded the adduct as a yellow solid (0.0185 g, 60%). mp: 216-218° C.; IR (CH₂Cl₂): ν=2993, 2944, 2925, 2866, 2157, 1707, 1568, 1464, 1270, 1088, 883; ¹H NMR (300 MHz, CDCl₃) δ 8.05 (m, 3H), 7.90 (s, 1H), 7.82 (d, J=8.4 Hz, 1H), 7.70 (m, 3H), 7.57 (m, 2H), 5.19 (s, 2H), 1.13 (m, 42H); ¹³C NMR (75 MHz, CDCl₃) δ 179.5 (C), 179.5 (C), 144.9 (C), 144.7 (C), 133.2 (C), 133.0 (C), 132.8 (C), 132.8 (C), 132.6 (C), 132.5 (CH), 132.0 (C), 131.9 (CH), 131.5 (CH), 130.9 (CH), 128.7 (C), 128.6 (CH), 128.3 (CH), 126.2 (CH), 126.1 (CH), 126.0 (CH), 125.9 (CH), 123.6 (C), 123.1 (C), 114.6 (CN), 114.6 (CN), 107.0 (C), 106.5 (C), 93.8 (C), 93.1 (C), 57.5 (C), 57.5 (C), 56.2 (CH), 56.1 (CH), 19.1 (CH₃), 19.1 (CH₃), 11.7 (CH), 11.7 (CH).

### Example 5

### 2,10-Bis(triisopropylsilylethynyl)-6,13-dihydropentacene

Lithium aluminium hydride (217 mg, 5.722 mmol, 20 eq) and aluminium chloride (381 mg, 2.861 mmol, 10 eq) were added by small portions at 0 degree (be careful, very exothermic) to a stirred solution of 2,10-bis(triisopropylsilylethynyl)-5,7,12,14-pentacenediquinone (200 mg, 0.286 mmol, 1 eq) in 5 mL of dried THF. The mixture was then refluxed overnight. The reaction was cooled to 0 degree and ethyl acetate was added dropwise to neutralize the excess of lithium aluminium hydride. The reaction mixture was diluted with ether and finally a saturated solution of sodium chloride was added until precipitation of the aluminium salts which were filtered on a plug of celite. The solvent was evaporated under reduced pressure and the resulting residue was purified by flash chromatography eluting with 6:1 petroleum ether/dichloromethane to afford 22 milligrams of desired compound as a white solid and a mixture of partially reduced compounds. The same procedure was repeated a second time on this mixture to give 40 milligrams of the desired compound (total: 62 mg, 34%); ¹H NMR (500 MHz, CDCl₃) δ 7.94 (br. s, 2H), 7.73 (s, 2H), 7.71 (s, 2H), 7.70 (d, J=8.5 Hz, 2H), 7.46 (dd, J=8.5, 1.4 Hz, 2H), 4.17 (s, 4H), 1.17 (s, 42H); ¹³C NMR (125 MHz, CDCl₃) δ 136.4, 136.2, 131.8, 131.7, 131.2, 128.4, 127.1, 125.0, 124.9, 120.2, 107.6, 90.5, 37.3, 37.1, 18.7, 18.6, 11.3, 11.2.

### Example 6

### Synthesis of Acetylenic Alcohol

n-Buli (1.2 ml, 3 mmol, 2.5 M in hexanes) was added dropwise to triisopropylsilyl acetylene (583 mg, 3.2 mmol) in dry THF (10 mL) under argon at 0° C. and mixture was stirred for half an hour. 2,9-bis-(tert-butyl-dimethylsilyloxy)-pentacene-6,13-dione (262.2 mg, 0.46 mmol) in THF (5 mL) was added and the mixture was stirred for 6 h at 0° C. The reaction was quenched with saturated NH₄Cl. The organic layer was separated and the aqueous layer was extracted with ether 3 times. The organic layers were combined and washed with brine, dried over Na₂SO₄. Solvent was removed and the residue was purified though chromatography to give the dialcohol (374 mg, 87%) as pale yellow oil.

¹HNMR (400 MHz, CDCl₃): 8.55 (s, 2H), 8.50 (s, 2H), 7.77 (d, J=8.8 Hz, 2H), 7.24 (d, J=2.0 Hz, 2H), 7.11 (dd, J=8.8, 2.4 Hz, 2H), 3.28 (s, 2H), 1.06 (m, 42H), 1.01 (s, 18H), 0.25(s, 12H). ¹³CNMR (100 MHz, CDCl₃): 154.3, 136.8, 134.5, 134.2, 129.6, 128.9, 125.7, 124.6, 123.1, 114.9, 109.6, 89.2, 69.7, 25.7, 18.7, 18.3, 11.3, −4.3. MS (ESI) Calcd 971.5 (M⁺+K), Found 971.4. IR (film) 2943, 2864, 1605, 1464, 1383, 1254

### Example 7

### 2,6,9,13-Tetrakis(2-triisopropylsilylethynyl)pentacene

2,9-Bis(trifluoromethylsulfonyloxy)-6,13-bis(2-triisopropylsilylethynyl)pentacene (136 mg, 0.15 mmol), Pd(PPh₃)₂Cl₂ (12 mg, 0.015 mmol) and CuI (6 mg, 0.03 mmol) were dissolved in a mixture of triethylamine (0.5 mL) and THF (5 mL) and degassed for 30 min. Triisopropylsilylacetylene (70 μL, 0.32 mmol) was added and the solution was stirred at 22° C. for 1 h. The mixture was then filtered through a pad a silica gel with ether as eluent and concentrated. The resultant organic residue was filter through a second silica gel pad using hexane as eluent. Removal of the solvent afforded 2,6,9,13-tetrakis(2-triisopropylsilylethynyl)pentacene (135.6 mg, 93%) as a blue solid. ¹H NMR (400 MHz, CDCl₃) 9.22 (s, 2H), 9.20 (s, 2H), 8.09(s, 2H), 7.87 (d, J=9.2, 2H),), 7.36 (dd, J=9.2, 1.2 Hz, 2H), 1.36 (s, 36H), 1.42-1.33 (m, 6H), 1.19 (s, 36H), 1.21-1.17 (m, 6H). ¹³C NMR (100 MHz, CDCl₃) 132.7, 131.6, 131.2, 131.0, 130.9, 128.6, 126.5, 126.4, 121.0, 118.8, 107.8, 107.7, 104.3, 93.1, 19.0, 18.7, 11.7, 11.4. IR (film) 2941, 2923, 2864, 2140, 2062, 1460, 1367.

While the invention has been described with reference to particular preferred embodiments thereof, it will be apparent to those skilled in the art upon a reading and understanding of the foregoing that numerous methods for substituted pentacene production, other than the specific embodiments illustrated are attainable, which nonetheless lie within the spirit and scope of the present invention. It is intended to include all such designs, assemblies, assembly methods, and equivalents thereof within the scope of the appended claims. With particular reference to the synthetic methods of the present invention, each method as claimed is intended to encompass obvious chemical equivalents thereof.

### REFERENCES

Kerdesky, F. A. J.; Ardecky, R. J.; Lakshmikantham, M. V.; Cava, M. C. J. Am. Chem. Soc. 1981, 103, 1992. Parakka, J. P.; Sandanandan, E. V.; Cava, M. P. J. Org. Chem. 1994, 59, 4308. Morris, J. L.; Becker, C. L.; Fronczek, F. R.: Daly, W. H.; McLaughlin, M. L. J. Org. Chem. 1994, 59, 6484.

1. A method for the preparation of a compound comprising at least one linear series of five fused carbon rings, the method comprising the steps of:
(a) providing an unsubstituted or substituted benzoquinone;
(b) providing an unsubstituted or substituted acyclic, cyclic, heterocyclic or ortho-quinodimethane diene;
(c) performing a double or stepwise cycloaddition reaction, optionally by a double Diels-Alder reaction, between the benzoquinone and the diene to generate a core structure comprising five fused carbon rings sequentially identified as rings A, B, C, D, and E.

2. The method of claim 1, further comprising at least one of the following optional steps:
(d) optionally performing a ring opening reaction to convert a bridged form of each of rings B and D to an unbridged form; and
(e) optionally performing an aromatization reaction or equivalent on the B, and D rings of the core structure;
(f) optionally replacing or adding selected substituents.
(g) optionally subjecting the compound to reducing conditions to generate a corresponding unsubstituted or substituted pentacene;
(h) optionally separating isomeric products, optionally by high performance liquid chromatography; and
(i) optionally performing a coupling reaction to link two or more core structures to generate an oligomeric compound comprising multiple units of said core structure, optionally linked via acetylene units at the 2 and 9 or 10 positions;
wherein any one or more of steps (d), (e), (f), (g), (h), and (i) where present may be performed in any order.

3. The method of claim 1, wherein in step (a) the benzoquinone has the general formula I: 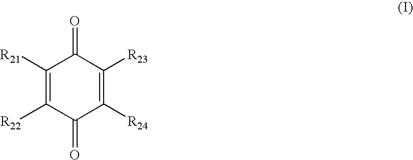
wherein optionally each R group is independently selected from the group consisting of hydrogen, an electron-withdrawing group, halogen, and a protonated amine.

4. The method of claim 1, wherein in step (b) the diene compound has the general formula IIa or IIb: 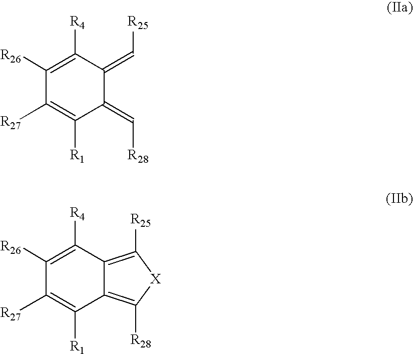
wherein each R group is H or any group that does not interfere with the capacity of the diene to undergo a cycloaddition reaction with benzoquinone, and X is C, O, S, or N, and optionally R₂₅ is a leaving group comprising OAlk, NAlk, or halide, wherein each Alk comprises an alkyl group of from 1 to 12 carbon atoms.

5. The method of claim 1, wherein in step (b) R₂₆ or R₂₇ comprises A-B, wherein A is a protective group, and B is a group to be protected, and wherein the method generates an compound of the formula III: 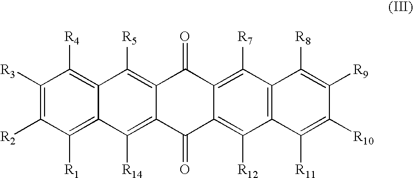
wherein R₂, and R₉ or R₁₀ are A-B, and each remaining R is each independently unsubstituted or substituted, the method optionally comprising the step of replacing each A-B at R₂, and R₉ or R₁₀ with an alternative substituent.

6. The method of claim 2, wherein the step of reducing generates a pentacene compound of formula IV: 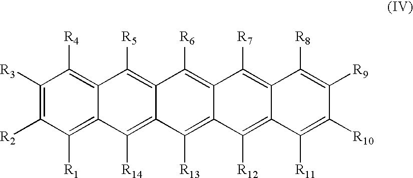
wherein R₂, and R₉ or R₁₀ are A-B, and optionally R₆ and R₁₃ are also A-B, where each A is a protective group and each B is a group to be protected, and each remaining R is each independently unsubstituted or substituted, the method optionally comprising the step of replacing each A-B at R₂, and R₉ or R₁₀ and optionally R₆ and R₁₃ with an alternative substituent.

7. The method of claim 6, wherein R₂, and R₉ or R₁₀ and optionally R₆ and R₁₃ comprise an unsubstituted or substituted group selected from acetylene, alkyl, aryl, heteroaryl, alkenyl, and alkynyl and preferably R₂ and R₉ or R₁₀ and optionally R₆ and R₁₃ comprise acetylene or a linker comprising one or more triple bonds, optionally substituted by halogen and/or triflate.

8. The method of claim 6, wherein each A-B comprises Si(R₃₀, R₃₁, R₃₂) wherein each of R₃₀, R₃₁, R₃₂ are independently selected from any group that in conjunction with Si acts to provide a protective group, preferably each A-B is independently selected from TMS, TES, TBS, TIPS, diphenyl tertiary butyl, OSi, OH, OTf, OTs, OMs, ONs, NSi, acetylene, phthalocyanine as a metal complex or free ligand, fullerene, Buckminsterfullerene C₆₀R₁₀₀, wherein R₁₀₀ is hydrogen or any substituant, or fullerene or phthalocyanine as a metal complex or free ligand, linked to the pentacene core either directly or via acetylene, and Buckminsterfullerene C₆₀R₁₀₀ linked to the pentacene core via acetylene;
and wherein optionally each B is O, S, Se, or N.

9. The method of claim 2 wherein in the step of replacing or adding selected substituents comprises replacing each A-B with Tf-O, halogen, or a substituent comprising a metal atom selected from Al, B, Cu, Co, Cr, Fe, Li, Mg, Ni, Pd, Pt, Si, Sn, Ti, and Zn, and optionally replacing each Tf-O with an acetylene group, or a group comprising a linker comprising one or more triple bonds.

10. A compound of formula III: 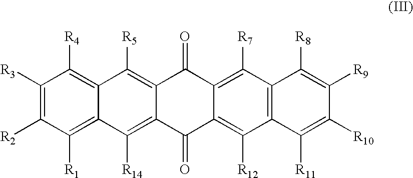
wherein R₁ to R₁₄ are each independently unsubstituted or substituted, the compound optionally comprising at least one substituent on each of the A and E rings of the core structure, and optionally further comprising at least one substituent on at least one of the B, C, or D rings of the core structure.

11. The compound of claim 10, the compound comprising substituents at least at the 2, and the 9 or 10 positions optionally comprising acetylene groups or attached to the core structure via a linker comprising one or more triple bonds.

12. The compound of claim 10, wherein each substituent is independently selected from hydroxyl, alkyl, alkoxy, alkenyl, alkynyl, aryl, heteroaryl, acetylene, halogen, triflate, fluoro, trifluoromethyl, nitro, hetroaryl, acetylene, and acetylene substituted with silyl, and wherein each substituant is optionally substituted by alkyl or halogen.

13. A compound of formula IV: 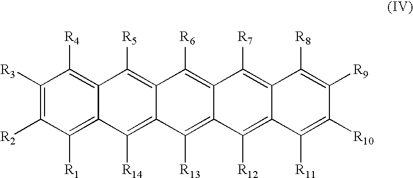
wherein each R group is independently unsubstituted or substituted, and optionally R₂ and R₉ or R₁₀ and optionally also R₆ and R₁₃ are A-B where each A is a protective group and each B is a group to be protected, or a compound of formula IV with each A-B replaced by a desired substituent, optionally with the proviso that when R₂ comprises an alkyl group, R₉ or R₁₀ does not also comprise an alkyl group, or when at least one of R₁, R₂, R₃, R₄, R₈, R₉, R₁₀, and R₁₁ are substituted with an electron-donating substituent, or a halogen, then the compound must include at least one further substituent at R₅, R₆, R₇, R₁₂, R₁₃, or R₁₄; the compound optionally comprising at least one substituent on each of the A and E rings, and optionally further comprising at least one further substituent on at least one of the B, C, or D rings of the core structure.

14. The compound of claim 13, the compound comprising substituents at least at the 2, and the 9 or 10 positions and optionally the 6 and 13 positions, optionally comprising acetylene groups, or optionally each being attached to the core structure via a linker comprising one or more triple bonds.

15. The compound of claim 13, wherein each A-B is replaced by a group independently selected from hydroxyl, alkyl, alkoxy, alkenyl, alkynyl, aryl, heteroaryl, acetylene, halogen, and triflate, or a group comprising alkyl or halogen.

16. Use of a compound obtainable by the method of claim 1 in the manufacture of a material suitable for use in ink-jet fabrication or as a component of an electronic device, optionally selected from the group consisting of an Organic Thin Film Semiconductor (OTFS), an Organic Field-Effect Transistor (OFET), an Organic Light Emitting Diode (OLED), a radio-frequency identification tag (RFID), a biosensor, a solar cell, and a component for solar energy conversion, said compound optionally exhibiting semiconductor properties.

17. Semiconductor material derived from processing of a compound obtainable by the method of claim 1.

18. An electronic device comprising the semiconductor material of claim 17, the device being optionally selected from an Organic Thin Film Semiconductor (OTFS), an Organic Field-Effect Transistor (OFET), an Organic Light Emitting Diode (OLED), a radio-frequency identification tag (RFID), a biosensor, a solar cell, and a component for solar energy conversion.

19. A method of generating a Diels-Alder reaction adduct of formula VII: 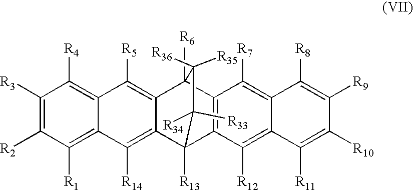
wherein each of R₁ to R₁₄ are as previously described, and each of R33 to R36 are preferably electron withdrawing groups etc.: by reaction of the compound of formula IV as defined in claim 13 with a dienophile optionally comprising sulfur dioxide, alkene dienophile, acyclic dienophile, cyclic dienophile, heterocyclic dienophile, or heteroatom dienophile.

20. A method of generating a compound of formula IV as defined in claim 13, the method comprising the step of:
causing the adduct of formula VII to undergo thermolysis to regenerate the compound of formula IV.

21. A compound of formula VII as described in claim 19, or an adduct thereof.

22. A method of generating a compound of formula VIIIa or VIIIb: 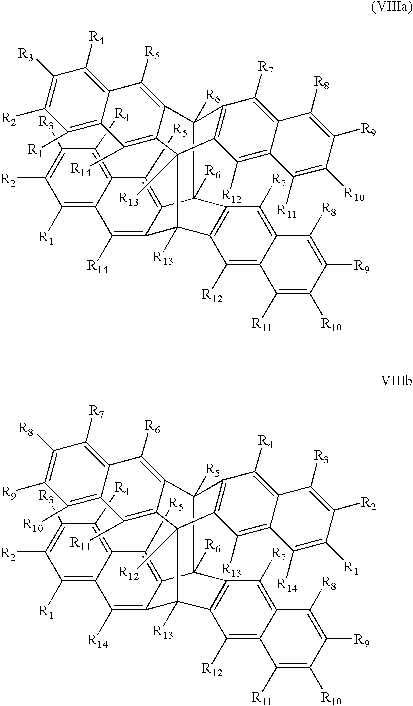
method comprising the step of: photochemical dimerization of the compound of formula IV as described in claim 13.

23. A compound of formula VIIIa or VIIIb as defined in claim 22.
